SEQUENCE LISTING (2 sequences)

SEQ ID NO: 1 - DNA, 20303 bp, Homo sapiens
ttcccatcaagccctagggctcctcgtggctgctgggagttgtagtctgaacgcttctatcttggcgagaagcgcctacgctccccctaccgagtcccgcggtaattcttaaagcacctgcaccgcccccccgccgcctgcagagggcgcagcaggtcttgcacctcttctgcatctcattctccaggcttcagacctgtctccctcattcaaaaaatatttattatcgagctcttacttgctacccagcactgatataggcactcaggaatacaacaatgaataagatagtagaaaaattctatatcctcataaggcttacgtttccatgtactgaaagcaatgaacaaataaatcttatcagagtgataagggttgtgaaggagattaaataagatggtgtgatataaagtatctgggagaaaacgttagggtgtggatattacggaaagccttcctaaaaaatgacatttaactgatgagaagaaaggatccagctgagagcaaacgcaaaagctttcttccttccacccttcatatttgacacaatgcaggattcctccaaaatgatttccaccaattctgccctcacagctctggcttgcagaattttccaccccaaaatgttagtatctacggcaccaggtcggcgagaatcctgactctgcaccctcctccccaactccatttcctttgcttcctccggcaggcggattacttgcccttacttgtcatggcgactgtccagctttgtgccaggagcctcgcaggggttgatgggattggggttttcccctcccatgtgctcaagactggcgctaaaagttttgagcttctcaaaagtctagagccaccgtccagggagcaggtagctgctgggctccggggacactttgcgttcgggctgggagcgtgctttccacgacggtgacacgcttccctggattgggtaagctcctgactgaacttgatgagtcctctctgagtcacgggctctcggctccgtgtattttcagctcgggaaaatcgctggggctgggggtggggcagtggggacttagcgagtttgggggtgagtgggatggaagcttggctagagggatcatcataggagttgcattgttgggagacctgggtgtagatgatggggatgttaggaccatccgaactcaaagttgaacgcctaggcagaggagtggagctttggggaaccttgagccggcctaaagcgtacttctttgcacatccacccggtgctgggcgtagggaatccctgaaataaaagatgcacaaagcattgaggtctgagacttttggatctcgaaacattgagaactcatagctgtatattttagagcccatggcatcctagtgaaaactggggctccattccgaaatgatcatttgggggtgatccggggagcccaagctgctaaggtcccacaacttccggacctttgtccttcctggagcgatctttccaggcagcccccggctccgctagatggagaaaatccaattgaaggctgtcagtcgtggaagtgagaagtgctaaaccaggggtttgcccgccaggccgaggaggaccgtcgcaatctgagaggcccggcagccctgttattgtttggctccacatttacatttctgcctcttgcagcagcatttccggtttctttttgccggagcagctcactattcacccgatgagaggggaggagagagagagaaaatgtcctttaggccggttcctcttacttggcagagggaggctgctattctccgcctgcatttctttttctggattacttagttatggcctttgcaaaggcaggggtatttgttttgatgcaaacctcaatccctccccttctttgaatggtgtgccccaccccccgggtcgcctgcaacctaggcggacgctaccatggcgtagacagggagggaaagaagtgtgcagaaggcaagcccggaggcactttcaagaatgagcatatctcatcttcccggagaaaaaaaaaaaagaatggtacgtctgagaatgaaattttgaaagagtgcaatgatgggtcgtttgataatttgtcgggaaaaacaatctacctgttatctagctttgggctaggccattccagttccagacgcaggctgaacgtcgtgaagcggaaggggcgggcccgcaggcgtccgtgtggtcctccgtgcagccctcggcccgagccggttcttcctggtaggaggcggaactcgaattcatttctcccgctgccccatctcttagctcgcggttgtttcattccgcagtttcttcccatgcacctgccgcgtaccggccactttgtgccgtacttacgtcatctttttcctaaatcgaggtggcatttacacacagcgccagtgcacacagcaagtgcacaggaagatgagttttggcccctaaccgctccgtgatgcctaccaagtcacagacccttttcatcgtcccagaaacgtttcatcacgtctcttcccagtcgattcccgaccccacctttattttgatctccataaccattttgcctgttggagaacttcatatagaatggaatcaggatgggcgctgtggctcacgcctgcactttggctcacgcctgcactttgggaggccgaggcgggcggattacttgaggataggagttccagaccagcgtggccaacgtggtgaatccccgtctctactaaaaaatacaaaaattagctgggcgtggtgggtgcctgtaatcccagctattcgggagggtgaggcaggagaatcgcttgaacccgggaggcagaggttgcagtgagccaagatcgtgccactacactccagcctgggcgacaagaacgaaactccgtctcaaaaaaaaggggggaatcatacattatgtgctcatttttgtcgggcttctgtccttcaatgtactgtctgacattcgttcatgttgtatatatcagtattttgctccttttcatttagtatagtccatcgattgtatatccgtccttttgatggccttttgagttgtttcccatttgcggttatgaaataaagctgctataaacattcttgtacaattctttttgtgatcatatgttttcgtgtttcttggagaaatacttaggaggggaattgtggaggaagtaaaaagtagctgtattttgaactttttcagaagctctgagttttccagagcggttgtaccattttacactccaactagcaaggtatgggagttattatggttgtgccacagccttccggacattaggtatgtcagtctttctaatgtggtatatccttgtggttgtaatttacagttctctattgactaaggatgttcagcattttttcatgtgcctattggccattcgtattttgtttgtaaagtagctcttcgagtcttttacctgttattttggtttttttgtttgtttttattgttcagttgtgggactgctttatactttctggatacaagtccttatcagatccatgagtcgtgaatgttttcttctgatctgttgcgggcctatttgtttgctttacagagtttacagaatcttaagaggagtggattaatcttttttatgttcagtatttgccttgtcctgtttaggacatctttttttttttttttaaccccagggtcatgaagatataatcttacattttcttttaggacctttatggtggtaagttttacagtaaggtccttaagccattaattaattcttaaaattaattgtttatggtgtgaggtgtaggagtcagtctctggtatctttcctgtatggaaatccagttattctgtctccacttgttgaaataggcttcctttctctactgaatgcttttaattttaattattttacagttggagtatagggctaccattttagtgctattttctttttttctttgttaatttttgagacagggactcacactgttgcccaggctagagtacaatggcacaatcaaggcttactgcagcctcgaacccctgggctcaagcagtcctctagcagcctcacgagtagctgggattactccaccacacccagctaactattttatttttttgtattgacaggatctcactatgttgcccaggctggtctcaaactgctggcctcaagctttcatcccatctcggcctcccaaagtgctgggattacaggtgtgagccaccatgcctgacctcttagtgctattttctatttatctcctctgttctctgctctctttaaacgttggaggaagaaacagtacccatcttacacaaactcttcagaaaacagaggaacagactgggcgcggtggctcatacctgtaatctcagcactttggtacgctgaggcaggggatcatttgaggtcgggagttcgagaccagcctggccaacacggcgaaaccccatctctactaaaatacaaaagtagctaggcgtgcaccatacctgtaatgccagttactcaggaggctgaggcacaagaatcccttgaacctgggaagcggaggttgcagtgagccgagattgcgccactgcactccagcctgggcaacagagtgagaccctgtctcagaaaaaaaaagaaagaaagaaaaaatagaggaatatttcccaacttgttttcgaagccaggataatcctggtaccaaaaccaaacaaggacattataagaaaagaaaatatagaccaatattcctgttagcatagacatgcaacagctaaccaattttagcaaaccaaacctggtaatatagaaaaaaggataaataggccagtcgcggtggctcacgcctgtaatcccagcactttgggaggctgaggcaggcagatcacttgaggtcaggagtttgagaccagcctgaccaacatggtgaaaccccgtttctaataaaaatacaaaaatcaggctgggcacggtggctcacgcctgtaatcccagcactttgggaggccgaggtgggcagatcacgaggtcaggagttcaagaccagcctgaccaatgtggtgaaacgccatctctactaaaaatacgaaaatcagccggtgtggtggcacctgcctgtaatcccagctactcaggaggctgaggcagaattgcttgaacccgggaggcagaggttgcagtgagccaagatcgtgccactgcactccagcctgggcgacagagcaagacttcatctcaaaaaaaaaaaaaattagctgggcatggtggtgggcacctgaaatcccagctactcgggagtctgaggcaggagaatcgcttgaacccaggaggcagaagttgcactgagctgggatcacaccattgcactccagcctgggcaacagagtgagactccatctcaaaaaaagaaaaagaaaaaggataaatacattctaaccaaataatgtttatctcatgattgtagctgattcaacattcaaaaattggcctggtgcagtagctcaggcctgtaatcccaacattttaggaggctgaggcaggaagatctcttgagcccaggatttcaagaccagcctgggcaacatagtcagactggtctttactggggggaaaaaaatcagtctgtgtaattcaccacattaacaaagggaaacataaaaaccctatgatcatttcaacagatgtagcaaaagcagttaatgatatcaacacatatgcatgattacaaaccaaccaacctcctagcaaactagggaaaggaaacttaactagtttgataacagggcgtccacagtcggagttccactagcagcatacataatggtagaaaactcagtgctgctgggggcggtggctcacgcctgtaatgccagcgctttgggaggcctaggcgggcggatcacgaggtcaggagatcgagactgtcctgactagcatgctgaaaccccgtctctactaaaaatacaaaaacaaaaaattagccgggcatggtggcgggcgcctatagtgccagctactcgggaggctgaggcgagagaatggcgtgaacccgggaggcggagcttgcagagcctagatcgtgccactgcactccagcctgggtgacagagtgagacttcgtctcaaaaaaaaaaaaaaaaaaaaaagaaaagaaaactcaacgctttttcctctaagatcaggaactagaaaaggatttgactctcacaacgttgataccatactggaggttttaaccaggcaagaaaaagaaataatgagggccgggtgcggtggctcaggcctgtaatcccagcactttgggaagccgagacgggtggatcacgaggtcaggagatcgagccatcctggctaacacggtgaaaccctgtctctactaaatatacaaaaaattagccgggcgtggtggcgggcgcctgtagtcccagctactcgggaggctgaggcaggagaatggcgtgaactcagggggcggagcttgcagtgagctgagatcgagccactgcactccagcctgggcgacagagcaagactgtgtctcaaaaaaaaaaaaagaaaaagaaataatgattagtggcccgatgtctcacgccagtaatcccagcactttgggaggccgaggtgggcagatcacctgaggtctggagttggagaccagcctgacaaagatggtgaaacctcgtctctattaaaatattaaaaaaatagccaggcgttggccgggtacagtggctcatgcctgtaaccccagcactttgggaggccgaggtgggtggatcacctgaggtcaggagttcaacaccagcctggccaacatggtgaaaccccatctctactaaaaatacaaaattagccgggcgtagtggcgggcgcctgtaatcccagctacttgggaggcttaggcaggagaatcgcttgaacctgggaggcggaggttgtagtgagccgagattgcaccattgcactccagcctgggtgacaaaagcaaaaactccgtctcaaaaaaaaaagaattagccaggggtagtggtgaacgcctgtagtcccagctactcaggaggcagaggcaggagaatcacttgaaccccggaggcagaggttgcagtgagccgagattgtcccattgcactccagcctaggcgagaagagcaaaattccatgtcaaaaaaaaaaaaaaaaaaggaaagaaaaaaaataacgattagaaaggaagaaatcaaacacattcacagccagtatgattctatacataccatggtcctaatggggccaggcgtggtggctcatgctgtaatcctagcacttttaggaggctgaggcaggtggcttccctgggaccagctggccaacatggtgaaaccccaactctaataaaaatacaaaaaatcagccaggcgtggtgagggcacctctaatcccagctactcaggaggctgaggcaggagaattgcttggacctgggaggcagaggttgcagtgagccgagatcgcgctattgcactccagcctgggcaacaagagtgaaactccggcagggtgtggtcttacgcctgtaatcccagcacttcgggaggctgagccaggccgatcacctgaggtcaggagtttgagaccaacctaacatggtgaaaccccgtctctactaaaaatacaagaattagctgggtgtagtggtgggcgcctgtaatcccagctacttgggaggctgagacagaagaattgcttgaacccaggaggtggaggttgcagtgagctgagatcatgccattgcacaccacgccgggcaacagagcgagattccgtctcaaaaaaaaaaaaaagatgaaactctatctcaaaaaaaaaaaaaagtcctaatggaaaatccataaaaagctaccaaaactaataaataaatatagcagggttgcaggttacagggcaatatagttatccctctatctgtaggggcttggttctgggactcctcacacaccaaacccacagatgtctaagtcccatatataagacggaatagtatttaacctacacatatcctcccatatagtttaaattatctagattacttacattacccccatacaatgaaaatgctaatgtacatgcaagtatgtatgtaagtacttgtactatattgtttagggaatcactggacagataggccttcaagactgataccagcagccactgttaagattctggtcaggcctgcccctgtttggggtctcagttgatctcattgccttcccacccagccaagggcacctgcatttctcttggctccctggccatttggaaggcctagttcagcctggcacatttgtatcctggcccactgatgctggtacccctgggaaggtcctgctctgaaaaacacggagattttagttgctactgaagatttgagagataaagacagggagacctgtctgtagacctgtgtccctccaagtgggattgagactttgggccccccatttcaggacagcacctcctggcctgttgactgaatagatccctgaaggaggtgtagttgcattttaggagtgggggtgggagcagtaccactgatccgcactaacaatcacacagttctctctagaataataatatagaacaagtgaaatagaacaattgcagaaagagctaacctttgttgagctcttactgtgtgcccagcactttcctcaactctacatttcccataatacatagagtactaggtaggcggggcttgggggctcacgcctgtaatcccagcactttaggaggccaaggggggtggatcacctgaggtcgggagttcaagaccagcctgactaacatggtgaaaccccgtctctactagaagtacaaaattagccaggtgtggtggcacatgcttgtagtcctagctactcagcaggctgaggcaggagaatcatttgaatccgggaggaggttgcagtaagcggagatagtgccactgtactccagcctgggcaataagagctgagactccgtctcaaaataaaataaaataaaataaaataaaataaaataaaataaaaaaagaaaagagcctgccattaaaggagctgtttggtaggggatgttttgtcagtgcaaacaacagaaaagtgggctgggcacagtggttcatgcctgtaatcccagcactttgggaggccaaggcgggcggatcacctgaagttgggagttcaagaccagcctgaccaatatggagaaaccccgtctctactaaaaatacaaaattagccgggcgcagtggccgatgcctgtaatcccagctactcgggaggctgaggcaggagaatcgcttgaacctgggaggcagaggttgcggtgagccgagatcgcaccattgcactccagcctggacgagagcaaaactctgtctcaaaaaaaaaaaaaaacagaaaagtgtaacaaacacttacagtaggcatgtttcttagcaaatctgatgacaaatttggcataaagaaagagagcatccctgaaaaaaaaaaaaagaaaaagaaagagagcatcctgcctgggcaacatagtgaaaccctgcctctacaaaaaaactcaaaaattggccgggtgcagtggctcacacctgtaatcccagcactttgggagtcggaggcgggaggatcacctgaggtcaggagttcgaaaccagcctggccaacatggcaaaaccccatctctactaaaaatacaaaaaattaatcaggcgcattggtgggcgcctgtaatcccagctactcaggaagttgaggcaagaggatcgcttgatactgggaggtggaggttacagtgagtcgagatcacaccactgcactctagcctgggtgacagggcgagactccgtctccaaaaaaaaaaagaaaaagaaaaagactaaaaaattagccaggcaggcctctgtggtcccagctacttgggaggctgaggcaggagaatcactgagcccaggagtggcaggctgtagtgagccatgattgcaccactgtaccctagcttgggcttcaaagcaagaccctgcctcaaaagaaaaaagaaagaaagaaagaacatggcgggccaggcacagtggctcacacctgtaatcccagcgctttgagaggccgaggcaggtggatcacaaggtcaggagttccacaccagcctggccaacatggtgaaaccctgtctctactaaaaatacaaaaaatcagcaggcagggtggtaggggcctgtaatcccagctactcgggaggctgaggcaggagaattgcttgaaaccagaaggcagaggttgcagtgagcctagactgcaccactgcactccagcctgggcgaaaagagccaaactccatctcaaaaaacaaacaaaaaaacaaaacaaaaaaaaacatggcagcctttgaaagcttgtctgggagaaggtgcgatgatggttgcataacttcgtgcaagatgctggtccacacaggggctgccccttgctctttctcgctctcttaacctctcatataacaggcttgtgtgttatgcacatttattgagcccaagcaggtgcaaggcattgtgatctaatactttggtcagcaagacaacaagatagatcactgccctgcccttaggaagtgtatatgctattagaggaaacagataaaataaacaaggaaaagtatcagacaatgtaagtgctatgagaatgcaaatgaggtgatgtgaattaaaataggatgacttaagtctgcacggaaggcccctacccccatgttcctggctagccaaggaaccaccagttgattagcagagaagggcagcccgtctagctagagcttttggggaagagggagtggttgttaagagatgagattaaagaagccgagacgggcccttcgtgagggggggttgtaatgcagggctgaggagtgtccgaagagaatgggcaggtgagcggtgagacagttgttcttccagaagctttgcagtgaaaggaatcaaagaaatggagccgtgtatcaggtggggaagggtgggggccaagggggtgtccttccccatacagagattgcaggctgagaatgactatatccttgttaacaggaggtgggagcagggcacggtagctcacacctgtaatcttggcactttaggaggcggaggcgggccgatcacctgaagtaaggagttcgagaccagcctggccaacatgcaaagccctgtctctactaaaaatacaaaaattagctgggtgtggtggtactcgcctgtaatcccagctactcgggagactgaggcaggagaatggcttgaacccggaaggtagaggttgcagtgagctgagatcatgccactgtgctccagcctaggtgacagagagagactccatctcaaaaaaaaaaaaaaatacaggaagggagttgggaatagggtgcacatttaggaagtcttggggatttagtggtgggaaggttggaagtccctctctgattgtcttttcctcaaagaagtgcatggctggtgtggggtggggcaggagtgcttgggttgtggtgaaacattggaagagagaatgtgaagcagccattcttttcctgctccacaggaagccgagctgtctcagacactggcatggtgttgggggaggggcctcctcctctgcaggcccaggtgacccagggttggaagcgtctcatgctggatccccacttttcctcttgcagcagccagactgccttccgggtcactgccatggaggagccgcagtcagatcctagcgtcgagccccctctgagtcaggaaacattttcagacctatggaaactgtgagtggatccattggaagggcaggccaccaccccgaccccaaccccagccccctagcagagacctgtgggaagcgaaaattcatgggactgactttctgctcttgtctttcagacttcctgaaaacaacgttctggtaaggacaagggttgggctgggacctggagggctgggggggctggggggctgaggacctggtcctctgactgctcttttcacccatctacagtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacggtcagttgccctgaggggctggcttccatgagacttcaatgcctggccgtatccccctgcatttcttttgtttggaactttgggattcctcttcacccttaggcttcctgtcagtgtttttttatagtttacccacttaatgtgtgatctctgactcctgtcccaaagttgaatattccccccttgaatttgggcttttatccatcccatcacaccctcagcatctctcctggggatgcagaacttttctttttcttcatccacgtgtattccttggcttttgaaaataagctcctgaccaggcttggtggctcacacctgcaatcccagcactctcaaagaggccaaggcaggcagatcacctgagccccaggagttcaagaccagcctgggtaacatgatgaaacctcgtctctacaaaaaaatacaaaaaattagccaggcatggtggtgcacacctatagtcccagccactcaggaggctgaggtgggaagatcacttgaggccaggagatggaggctgcagtgagctgtgatcacaccactgtgctccagcctgagtgacagagcaagaccctatctcaaaaaaaaaaaaaaagaaaagctcctgaggtgtagacgccaactctctctagctcgctagtgggttgcaggaggtgcttacacatgtttgtttctttgctgccgtgttccagttgctttatctgttcacttgtgccctgactttcaactctgtctccttcctcttcctacagtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtgagcagctggggctggagagacgacagggctggttgcccagggtccccaggcctctgattcctcactgattgctcttaggtctggcccctcctcagcatcttatccgagtggaaggaaatttgcgtgtggagtatttggatgacagaaacacttttcgacatagtgtggtggtgccctatgagccgcctgaggtctggtttgcaactggggtctctgggaggaggggttaagggtggttgtcagtggccctccgggtgagcagtaggggggctttctcctgctgcttatttgacctccctataaccccatgagatgtgcaaagtaaatgggtttaactattgcacagttgaaaaaactgaagcttacgaggctaagggcctcccctgcttggctgggcgcagtggctcatgcctgtaatcccagcactttgggaggccaaggcaggcggatcacgaggttgggagatcgagaccatcctggctaacggtgaaaccccgtctctactgaaaaatacaaaaaaaaattagccgggcgtggtgctgggcacctgtagtcccagctactcgggaggctgaggaaggagaatggcgtgaacctgggcggtggagcttgcagtgagctgagatcacgccactgcactccagcctgggcgacagagcgagattccatctcaaaaaaaaaaaaaaaaggcctcccctgcttgccacaggtctccccaaggcgcactggcctcatcttgggcctgtgttatctcctaggttggctctgactgtaccaccatccactacaactacatgtgtaacagttcctgcatgggcggcatgaaccggaggcccatcctcaccatcatcacactggaagactccaggtcaggagccacttgccaccctgcacactggcctgctgtgccccagcctctgcttgccgctgacccctgggcccacctcttaccgatttcttccatactactacccatccacctctcatcacatttccggcgggaatctccttactgctcccactcagtttccttttctctggctttgggacctcttaacctgtggcttctcctcccacctcctggagctggagcttaggctccagaaaggacaagggtggttgggagtagatggagcctggttttttaaatgggacaggtaggacctgatttccttactgcctcttgcttctcttttcctatcctgagtagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgaggtaagcaagcaggacaagaagcggtggaggagaccaagggtgcagttatgcctcagattcacttttatcacctttccttgcctctttcctagcactgcccaacaacaccagctcctctccccagccaaagaagaaaccactggatggagaatatttcacccttcaggtactaagtcttgggacctcttatcaagtggaaagtttccagtctaacactcaaaatgccgttttcttcttgactgttttacctgcaattggggcatttgccatcagggggcagtgatgcctcaaagacaatggctcctggttgtagctaactaacttcagaacaccaacttataccataatatatattttaaaggaccagaccagctttcaaaaagaaaatagttaaagagagcatgaaaatggttctatgactttgcctgatacagatgctacttgacttacgatggagttacttctgataactcgtcgtaagttgaaatattgaaatattgtaagttgaaaatggatttaatacacctaatctaaggaacatcatagcttagcctagcctgctttttttttttttttttttttggagacagagtctcactctgctacccaggctggagtgcagtggcgggatctcggctcactgcaacctccgccttctgggttcaagcgattctcctgcctcagcccactgagtagctgggattacaggcacctgccccgacgcccagctaattttttgttatttatttcctttttttttagtagagatagaatttcaccatgttggccaggctagtctcgaactcctgaccttgtgatctgcctgccttggcctcccaaagtgctgggattacaggcgtgagccaccgcacctggcctgcctagcctacttttattttatttttaatggagacagcatcttgctctgttgcccaggctggattacagtgatgtgatcatagctcattataccctcctgggctcaagcaatccccctaactctgcctccccagtagctaggaccacaggcatacaccaccatacccagctaatttttaaaattttttgtagatagatagagtctcactatgttgcccaggctggtctctagcctacttttttgagacaaggtcttgctctgtcacccaggctggatagagtgcagtagtgcagtcacagctcactgcagcctccacctcccaggctccatccatcctcccagctcagcctcccaagttgcttcaactacaggcctgcaccaccatgcctggctaatttttatttatttatttttattttattttattttattttttgagactcagtctcactctgtcgccttaggctggagtgcagtggcatgatctcggctcactgctaacctctgcctcctgggtttcaagtgattctcctgcctcagcctcccgaatagctaggactacaagcgcctgctaccacgcccggctaatttgtgtatttttagtagagacagggtttcaccatgttggccaggctggtctcgaacttctgaccatgtgatcgccgcctcggcctcccaaagtgctgggattacaggtgtgagccaccacgcccggctaatttttatttatttatttaaagacagagtctcactctgtcactcaggctagagtgcagtggcaccatctcagctcactgcagccttgacctccctgggctccggtgatttcaccctcccaagtagctaggactacaggcacatgccacgacacccagctaattttttattttctgtgaagtcaaggtcttgctacgttgcccatgctggtatcaaacccctgggctcaatcaatccttccacctcagcctccccaagtattggggttacaggcatgagctaccacactcagccctagcctacttgaaacgtgttcagagcatttaagttaccctacagttgggcaaagtcatctaacacaaagccctttttatagtaataaaatgttgtatatctcatgtgatttattagatattgttactaaaagtgagaaacagcatggttgcatgaaaggaggcacagtcgaagccaggcacagcctgggcgcagagcgagactcaaaaaaagaaaaggccaggcgcactctcacgcctgtaatcccagcatttcgggaggctgaggcgggtggatcacctgaggtcaggagttcaagaccagcctagccaacatggtgaaaccccgtctctactaaaatacaaaaattaaccgggcgtgatggcaggtgcctgtaatcccagctacttgggaggctgaggcaggagaatcgcttgaaccaggaggcggaggttgcagggagccaagacggcgccactgcactccagcctgggcgatagagtgagactccgtctcagaaaaaaaagaaaagaaacgaggcacagtcgcatgcacatgtagtcccagttacttgagaggctaaggcaggaggatctcttgagcccaagagtttgagtccagcctgaacaacatagcaagacatcatctctaaaatttaaaaaagggccgggcacagtggctcacacctgtaatcccagcactttgggaggtggaggtgggtagatcacctgacgtcaggagttggaaaccagcctggctaacatggtgaagccccatctctactaaaaacacaaaaattagccagtgtgagacacgttgagtccacgtactcggaggctgaggcacaagaatcacttgaaccccagaggcggagattcgaatcagccaagattgcaccattgcactcccgcctgggcgacgagagtgagaccccatctcaaaataaataaataaatatttttaaaagtcagctgtataggtacttgaagtgcagtttctactaaatcgatgttgcttttgatccgtcataaagtcaaacaattgtaacttgaaccatcttttaactcaggtactgtgaatatacttacttctccccctcctctgttgctgcagatccgtgggcgtgagcgcttcgagatgttccgagagctgaatgaggccttggaactcaaggatgcccaggctgggaaggagccaggggggagcagggctcactccaggtgagtgacctcagccccttactggccctactcccctgccttcctaggttggaaagccataggattccattctcatcctgccttcatggtcaaaggcagctgaccccatctcattgggtcccagccctgcacagacatttttttagtcttcctccggttgaatcctataaccacattcttgcctccacgtagtatccacagaacatccaaacccagggacgagtgtggatacttctttgccattctccgccaactccccagcccagagctggagggtctcaaggggcctaataattgtgtaatactgaatacagccagagtttcaggtcatatactcagccctgccatgcaccggcaggtcctaggtgacccccgtcaaactcagtttccttatatataaaatggggtaagggggccgggcgcagtggctcacgaatcccacactctgggaggccaaggcgagtggatcacctgaggtcgggagtttgagcccagcctgaccaacatggagaaaccccatctctactaaaaatacaaaagtagccgggcgtggtgatgcatgcctgtaatcccagctacctactcgggaggctgaggcaggagaatcgcttgaacccgggaggcagaggttgcggtgagctgagatctcaccattacactccagcctgggcaacaagagtgaaactccgtctcaaaaaagtataataaagtaaaatggggtaagggaagattacgagactaatacacactaatactctgaggtgctcagtaaacatatttgcatggggtgtggccaccatcttgatttgaattcccgttgtcccagccttaggcccttcaaagcattggtcagggaaaaggggcacagaccctctcactcatgtgatgtcatctctcctccctgcttctgtctcctacagccacctgaagtccaaaaagggtcagtctacctcccgccataaaaaactcatgttcaagacagaagggcctgactcagactgacattctccacttcttgttccccactgacagcctccctcccccatctctccctcccctgccattttgggttttgggtctttgaacccttgcttgcaataggtgtgcgtcagaagcacccaggacttccatttgctttgtcccggggctccactgaacaagttggcctgcactggtgttttgttgtggggaggaggatggggagtaggacataccagcttagattttaaggtttttactgtgagggatgtttgggagatgtaagaaatgttcttgcagttaagggttagtttacaatcagccacattctaggtaggtaggggcccacttcaccgtactaaccagggaagctgtccctcatgttgaattttctctaacttcaaggcccatatctgtgaaatgctggcatttgcacctacctcacagagtgcattgtgagggttaatgaaataatgtacatctggccttgaaaccaccttttattacatggggtctaaaacttgacccccttgagggtgcctgttccctctccctctccctgttggctggtgggttggtagtttctacagttgggcagctggttaggtagagggagttgtcaagtcttgctggcccagccaaaccctgtctgacaacctcttggtcgaccttagtacctaaaaggaaatctcaccccatcccacaccctggaggatttcatctcttgtatatgatgatctggatccaccaagacttgttttatgctcagggtcaatttcttttttcttttttttttttttttttctttttctttgagactgggtctcgctttgttgcccaggctggagtggagtggcgtgatcttggcttactgcagcctttgcctccccggctcgagcagtcctgcctcagcctccggagtagctgggaccacaggttcatgccaccatggccagccaacttttgcatgttttgtagagatggggtctcacagtgttgcccaggctggtctcaaactcctgggctcaggcgatccacctgtctcagcctcccagagtgctgggattacaattgtgagccaccacgtggagctggaagggtcaacatcttttacattctgcaagcacatctgcattttcaccccacccttcccctccttctccctttttatatcccatttttatatcgatctcttattttacaataaaactttgctgccacctgtgtgtctgaggggtgaacgccagtgcaggctactggggtcagcaggtgcaggggtgagtgaggaggtgctgggaagcagccacctgagtctgcaatgagtgtggactggggggcccagtgcccgggttccgggaggggaacaaaggctggagactgggtcagtctgcgggctgcatgacaacaagggagggggtggctccattcataactcaggaaccaaccgtccctcctcccctccgcccacggctggcacaaggttctctgcctcccctgcttctaggattgggctgcttccccctcggcagcctctcaccaaggattacgggatttaaatgtcgtgatttacgaaggctgagcctccagggtggccatcttcgtccatcagaagtggcaggatacctgggttccaagggaacagggtgg

SEQ ID NO: 2 - protein, 393 aa, Homo sapiens [sequence omitted]

(19) United States
(12) Patent Application Publication
(10) Pub. No.: US 2003/0223967 A1
(43) Pub. Date: Dec. 4, 2003

(54) Adenoviral p53 gene transfer in the prevention and treatment of injury-induced vascular smooth muscle cell proliferation

(76) Inventors: Louis Zumstein, Houston, TX (US); Sunil Chada, Missouri City, TX (US)

Correspondence Address: FULBRIGHT & JAWORSKI L.L.P., 600 CONGRESS AVE., SUITE 2400, AUSTIN, TX, 78701, US

(21) Appl. No.: 10/434,693

(22) Filed: May 9, 2003

Related U.S. Application Data
(60) Provisional application No. 60/379,075, filed on May 9, 2002.

(51) Int. Cl. A61K 48/00; C12N 7/00; C12N 15/86

(52) U.S. Cl. 424/93.2; 435/235.1; 435/456

(57) ABSTRACT

The present invention provides for the use of expression constructs encoding the tumor suppressor p53 for the prevention or treatment of vascular stenosis caused by vascular smooth muscle cell (VSMC) proliferation and/or migration in response to vascular trauma. This p53 therapy may be used in conjunction with other therapies including secondary gene therapy, anti-thrombotics or anti-inflammatory agents.

### BACKGROUND OF THE INVENTION

The present application claims priority to co-pending U.S. Provisional Patent Application Serial No. 60/379,075 filed on May 9, 2002. The entire text of the above-referenced disclosure is specifically incorporated herein by reference without disclaimer.

1. Field of the Invention

The present invention relates generally to the fields of cardiology, vascular biology, and molecular biology. More particularly, it concerns the use of gene therapy to address pathological proliferation of coronary artery smooth muscle cells following injury.

2. Description of Related Art

Vascular interventions intended to restore blood flow to narrowed or obstructed blood vessels, including angioplasty and bypass grafting, cause vessel injury. This injury often leads to intimal thickening, a process in which normally quiescent vascular smooth muscle cells (VSMC) such as coronary artery smooth muscle cells, and possibly other cells in the vascular wall, adopt a secretory phenotype associated with cell cycle activation, mitosis, migration, and prolonged secretion of extracellular matrix (“fibrosis”). Together with late thrombosis, this exaggerated “healing response,” resulting in intimal hyperplasia, is a major cause of graft and angioplasty failure, despite a variety of previously described treatment strategies. Mechanical strategies such as stenting, combined with the use of endoluminal radiation (“brachytherapy”) or stent-based drug delivery, have shown some success but have not eliminated this problem.

In addition, systemic pharmacological methods have not proven effective in preventing restenosis, in part because the complex interplay of inflammation, thrombosis, cell proliferation, VSMC migration, and fibrosis at the site of injury remains only incompletely understood. Novel treatment approaches are currently undergoing testing, and some gene therapy approaches have shown promise in this area. Gene transfer approaches to deter intimal hyperplasia have in general focused on cell cycle inhibition, induction of apoptosis, reduction of inflammation, inhibition of thrombosis, or a combination of these approaches (Wattanapitayakul and Bauer, 2000; Channon and Annex, 2000; Mann, 2000; Smith and Walsh, 2000; Ehasn and Mann, 2000; Yla-Herttuala and Martin, 2000). However, there remains a need for improved methods of controlling unwanted VSMC proliferation, such as coronary artery smooth muscle cell proliferation, particularly in the context of vascular stenosis.

### SUMMARY OF THE INVENTION

Thus, in accordance with the present invention, there is provided a method for reducing vascular smooth muscle cell (VSMC) proliferation, such as coronary artery smooth muscle cell proliferation, comprising introducing into the cell a first expression cassette comprising a promoter active in the cell and a first nucleic acid segment encoding p53, wherein the nucleic acid segment is under the transcriptional control of the promoter. The expression cassette may be comprised within a viral vector selected from the group consisting of an adenovirus, a retrovirus, an adeno-associated virus, a vaccinia virus, a herpesvirus or a polyoma virus. The adenoviral vector may be a replication defective adenoviral vector which may lack at least a portion of the E1 region, or does not encode functional E1A or E1B proteins, or may lack the entire E1 region.

In some embodiments of the invention, the expression vector may be a non-viral expression vector which may be encapsulated in a lipid delivery complex.

It is contemplated that the promoter as discussed above may be a constitutive promoter, or a promoter that is preferentially active in a vascular smooth muscle cell.

In some embodiments, the expression construct may further comprise a polyadenylation signal and/or an origin of replication, or a second nucleic acid segment encoding an anti-proliferative protein other than p53. The anti-proliferative protein may comprise a tumor suppressor, an inducer of apoptosis, a cell cycle regulator, a tissue factor inhibitor, an inhibitor of a platelet-borne group of proliferative or pro-migratory molecules, or a toxin.

In particular embodiments, the anti-proliferative protein may be a tumor suppressor such as PTEN or Rb. In another particular embodiment, the anti-proliferative protein may be an inducer of apoptosis such as Bcl-2, Bax, Bad, or Bid. In yet another particular embodiment, the anti-proliferative protein may be a cell cycle regulator such as E2F-1. In still yet another particular embodiment, the anti-proliferative protein may be an enzyme such as cyclooxygenase-1 (COX-1), or tissue factor pathway inhibitor (TFP-1), endothelial nitric oxide synthase (eNOS), inducible nitric oxide (iNOS), or inhibitors of matrix metalloprotein (TIMP-1 or TIMP-2).

It is also contemplated that the first and second nucleic acid segments may be under the control of the same promoter, separated by an internal ribosome entry site, or under the control of different promoters.

In some embodiments of the invention, the vascular smooth muscle cell may be located in a subject such as a human, in an uninjured early atherosclerotic lesion, in a transplanted organ, in a graft such as a vein/artery graft, in a vein or artery that has been or will be subject to trauma such as angioplasty. It is contemplated that the trauma may comprise insertion of a stent, a graft or a conduit.

In some embodiments of the present invention, it is contemplated that an anti-inflammatory compound, an inhibitor of thrombosis, a fibrolytic agent, or endoluminal radiation may be administered to a subject.

In other embodiments of the present invention, one may introduce into the cell a second expression cassette comprising a promoter active in the cell and a second nucleic acid segment encoding an anti-proliferative protein other than p53, wherein the nucleic acid segment is under the transcriptional control of the promoter.

The anti-proliferative protein may comprise a tumor suppressor, an inducer of apoptosis, a cell cycle regulator, a tissue factor inhibitor, an inhibitor of a platelet-borne group of proliferative or pro-migratory molecules, or a toxin.

It is contemplated in the present invention that the expression cassette may be introduced intravenous or by intraarterial delivery. In further embodiments, introducing the expression construct may comprise restricting blood flow away from the region of the delivery by applying a tourniquet, a cuf, or a catheter (e.g., injection method catheters) to the human subject. In particular embodiments, the expression cassette may be coated or impregnated on the surface of an intravascular device prior to insertion into the human subject.

The present invention further provides a method for reducing vascular smooth muscle cell (VSMC) migration comprising introducing into a cell an expression cassette comprising a promoter active in the cell and a first nucleic acid segment encoding p53; the nucleic acid segment is under the transcriptional control of the promoter.

The present invention also provides a method for reducing an atherosclerotic lesion in a subject comprising introducing into cells of the lesion an expression cassette comprising a promoter active in the cell and a first nucleic acid segment encoding p53; the nucleic acid segment is under the transcriptional control of the promoter.

In still yet another embodiment, the present invention provides a method for preventing development of an atherosclerotic lesion in a subject comprising introducing into vascular smooth muscle cells of the subject an expression cassette comprising a promoter active in the cell and a first nucleic acid segment encoding p53; the nucleic acid segment is under the transcriptional control of the promoter.

In still yet another embodiment, the present invention provides method for of inhibiting intimal hyperplasia in a subject comprising introducing into a vascular smooth muscle cells of the subject an expression cassette comprising a promoter active in the cell and a first nucleic acid segment encoding p53; the nucleic acid segment is under the transcriptional control of the promoter.

Hyperplasia or tumor cell proliferation is promoted by the formation of new blood vessels (angiogenesis). Angiogenesis, a complex multi-step process, involves endothelial cell (EC) migration, proliferation and differentiation into vascular tubes (i.e., tube formation). By inhibiting tube formation (angiogenesis), local and systemic tumor cell proliferation may be inhibited. Thus, the present invention also contemplates a method of inhibiting tube formation in a cell comprising introducing into a cell, such as a vascular smooth muscle cell, a first expression cassette comprising a promoter active in said cell and a first nucleic acid segment encoding p53, wherein said nucleic acid segment is under the transcriptional control of said promoter.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Transduction of normal human cells.

FIG. 2. Inhibition of HUVEC proliferation.

FIG. 3. Decrease of HUVEC viability (FACS).

FIG. 4. Increase of HUVEC apoptosis (FACS).

FIG. 5. Decrease of HUVEC viability (FACS).

FIG. 6. Increase of HUVEC apoptosis and necrosis (FACS).

FIG. 7. Decrease of HUVEC viability (trypan blue exclusion).

FIG. 8. Decrease of quiescent HUVEC viability (trypan blue exclusion).

FIG. 9. Decrease of quiescent HUVEC viability (FACS).

FIG. 10. Increase of quiescent HUVEC apoptosis and necrosis (FACS).

FIG. 11. Inhibition of HMEC proliferation, day 2 post-infection.

FIG. 12. Inhibition of HMEC proliferation, day 3 post-infection.

FIG. 13. Inhibition of HMEC proliferation.

FIG. 14. Decrease of HMEC viability (FACS).

FIG. 15. Increase of HMEC apoptosis (FACS).

FIG. 16. Effects on MJ90 proliferation.

FIG. 17. Decrease of HCASMC viability (lot 16645, trypan blue exclusion).

FIG. 18. Decrease of HCASMC viability (lot OF1129, trypan blue exclusion).

FIG. 19. HCASMC apoptotic cells (lot 16645 early and late apoptotic cells).

FIG. 20. HCASMC apoptotic cells (lot OF1129 early and late apoptotic cells).

### DESCRIPTION OF ILLUSTRATIVE EMBODIMENTS

Vascular interventions intended to restore blood flow to narrowed or obstructed blood vessels cause vessel injury and lead to intimal thickening, a process in which normally quiescent vascular smooth muscle cells (VSMC), such as coronary artery smooth muscle cells, proliferate and narrow vascular passages. Various treatment strategies have been attempted, such as stenting, endoluminal radiation (“brachytherapy”), and stent-based drug delivery. While these have shown some success, they have not eliminated the problem. Newer systemic pharmacological methods have not proven effective in preventing restenosis, in part because the complex interplay of inflammation, thrombosis, cell proliferation, VSMC migration. Thus, the need remains for new methods to attack this pathologic condition. One such method as is proposed by the current invention, involves adenoviral gene therapy.

There are several reports in the literature on the effect of Ad5CMV-p53 (INGN 201) or other adenoviral vectors expressing p53 on normal human cells. Regarding studies with Ad5CMV-p53 (INGN 201), Zhang et al. (1995), found that the proliferation of normal human bronchial epithelial cells was not affected by Ad5CMV-p53 after infection at up to 100 pfu/cell (˜2,000 vp/cell); at this MOI, greater than 80% of the cells were expressing high levels of p53 protein, and the cancer cell line H1299 was completely blocked for proliferation. Kawabe et al. (2001), found that the normal human lung fibroblast line MRC-9 was completely resistant to the apoptosis inducing effects of Ad5CMV-p53 seen in two lung cancer cell lines (A549 and H322).

For adenoviral-p53 vectors other than Ad5CMV-p53, (Blagosklonny et al., 1998 and Kim et al., 2001) it was found that normal human skin fibroblasts (1784 and HS27) were not affected by an adenoviral-p53 vector at MOIs up to 50 to 100 pfu/cell (˜1,000 to 2,000 vp/cell), while the normal human lung fibroblast line WI-38 was sensitive (50% inhibition of cell viability at 10 pfu/cell, ≅200 vp/cell). Additionally, Liu et al. (2000), concluded that an adenoviral-p53 vector is a “potent inducer of apoptosis” in multiple myeloma cells, but is “not overtly toxic to normal hematopoietic stem cells or normal lymphocytes.” D'Orazi et al. (2000), compared the effects of an adenoviral-p53 vector on normal C₂C₁₂ mouse myoblasts and their Ras transformed counterpart, C₂-ras. The C₂C₁₂ cells were less sensitive to the action of the adenoviral-p53 than the transformed C₂-ras cells, and the mechanisms potentially responsible for the difference included: (a) lower p53 protein levels in C₂C₁₂ cells (different p53 half lives); (b) greater CMV promoter activity in the transformed cell line; and (c) differential transcriptional activity of the expressed p53 protein (p53 was not activated in the non-transformed cells). The relevance of the results of D'Orazi et al. (2000), to normal human cells is unclear. Several authors note that normal fibroblasts are difficult to infect (Blagosklonny et al., 1998, and Kim et al., 2002). More directly, clinical trials which involved intensive treatment of normal wound beds after the surgical removal of HNSCC tumors found no effect of INGN 201 on wound healing (Clayman et al., 1998).

I. The Present Invention

The present invention provides a replication-impaired adenoviral (derived from adenovirus sterotype 5 (Ad5)) vector that is E1-deleted and contains the p53 gene designated as ADVEXIN®, INGN 201, or Ad5CMV-p53. INGN 201 also has mutations which abrogate the expression of four of the E3 proteins (Bett et al., 1995); these proteins normally ameliorate the host immune response to adenoviral infection (Shenk, 1996), so the virus is presumed to be even further crippled in vivo. The p53 expression cassette has been inserted at the site of the E1-deletion, and drives high level p53 protein expression (Zhang et al., 1993).

Though integrated adenoviral DNA has not been found in the human genome, E-deleted AdS vectors can integrate after infection of tissue culture cells; they seem to insert into the genome randomly and as full length or nearly full length adenoviral DNA (Harui et al., 1999; Van Doren et al., 1984 and Hillgenberg et al., 2001). The mechanism of E1-deleted adenoviral vector integration is unclear, though a non-homologous end joining mechanism of insertion has been suggested (Hillgenberg et al., 2001). Several studies have shown that E1-deleted adenoviral vectors integrate into cultured rat, mouse, simian or human cell lines with a frequency of 10⁻² to 10⁻⁵ viral integrations per cell (Harui et al., 1999; Van Doren et al., 1984 and Hillgenberg et al., 2001). In order to enhance the integration frequency and long-term stability of transgene expression of adenoviral transfected plasmid DNA, a variety of DNA damaging agents that include but is not limiting to agents such as cisplatin, doxorubicin and fluorouracil; and ionizing radiation such as gamma radiation, X-ray and UV radiation may be used.

A number of E1-deleted adenoviral vectors have been used in human clinical trials and vector related serious adverse effects are rarely noted (e.g., Stewart et al., 1999, Schuler et al., 1998; Sterman et al., 1998). It has also been shown that intratumoral routes of administration lead to systemic exposure (Clayman et al., 1998). Over 500 patients have been treated with escalating doses of INGN 201, and vector related serious adverse events are rare (Clayman et al., 1998, 1999; Roth et al., 1998; Swisher et al., 1999; Weill et al., 2000; Nemunaitis et al., 2000; Merritt et al., 2001). These studies have involved over 20 completed and ongoing clinical trials, and routes of administration have included intratumoral, intravenous (i.v.), and intraperitoneal administration. INGN 201 has been used to treat numerous different types of cancer including lung, head and neck, glioma, ovarian, prostate, and breast.

INGN 201 can inhibit tumor cells in culture and significantly reduce tumor growth in animal models of human cancers. INGN 201 is safe and has low toxicity. INGN 201 has little or no effect on normal human fibroblasts or human mammary epithelial cells in vitro. In human umbilical vein endothelial cells (HUVEC), INGN 201 caused complete inhibitiomof tube formation. The present invention therefore provides a method of inhibiting proliferation of vascular smooth muscle cells such as human coronary artery smooth muscle cells, using INGN 201. Furthermore, the present invention provides a therapeutic modality for the treatment of vascular occlusive diseases such as peripheral arterial disease (PAD), coronary artery disease (CAD), and restenosis, as vascular smooth muscle cell proliferation and migration is believed to be necessary for these vascular occlusive disease occurrences.

II. p53

p53 currently is recognized as a tumor suppressor gene. High levels of mutant p53 have been found in many cells transformed by chemical carcinogenesis, and ultraviolet radiation. The p53 gene is a frequent target of mutational inactivation in a wide variety of human tumors and is already documented to be the most frequently-mutated gene in common human cancers. It is mutated in over 50% of all human tumors (Oren et al., 1999) making it the most frequent target for genetic alterations in cancer.

The p53 gene encodes a 393-amino acid phosphoprotein that can form complexes with proteins such as SV40 large-T antigen and adenoviral E1B. The protein is found in normal tissues and cells, but at concentrations which are minute by comparison with transformed cells or tumor tissue. Interestingly, wild-type p53 appears to be important in regulating cell growth and division. Overexpression of wild-type p53 has been shown in some cases to be anti-proliferative in human tumor cell lines. Thus, p53 can act as a negative regulator of cell growth (Weinberg, 1991) and may directly suppress uncontrolled cell growth or indirectly activate genes that suppress this growth. Thus, absence or inactivation of wild-type p53 may contribute to transformation. However, some studies indicate that the presence of mutant p53 may be necessary for full expression of the transforming potential of the gene.

Wild-type p53 is recognized as an important growth regulator in many cell types. Missense mutations are common for the p53 gene and are essential for the transforming ability of the oncogene. A single genetic change prompted by point mutations can create tumor promoting p53, in as much as mutations in p53 are known to abrogate the tumor suppressor capability of wild-type p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant-negative alleles (Weinberg, 1991).

Casey and colleagues have reported that transfection of DNA encoding wild-type p53 into two human breast cancer cell lines restores growth suppression control in such cells (Casey et al., 1991). A similar effect also has been demonstrated on transfection of wild-type, but not mutant, p53 into human lung cancer cell lines (Takahasi and Sawasaki, 1992). p53 appears dominant over the mutant gene and will select against proliferation when transfected into cells with the mutant gene. Normal expression of the transfected p53 does not affect the growth of normal or non-malignant cells with endogenous p53. Thus, such constructs might be taken up by normal cells without adverse effects. It is thus proposed that the treatment of p53-associated cancers with wild-type p53 will reduce the number of malignant cells or their growth rate.

III. Expression Constructs

In order to deliver p53 to cells, it is desirable to introduce a nucleic acid segment coding for p53 into an expression vector. The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et al. (1988) and Ausubel et al. (1994), both incorporated herein by reference.

The term “expression vector” refers to a vector containing a nucleic acid sequence or “cassette” coding for at least part of a gene product capable of being transcribed and “regulatory” or “control” sequences, which refer to nucleic acid sequences necessary for the transcription and possibly translation of an operably linked coding sequence in a particular host cell. In addition to control sequences that govern transcription and translation, expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence. Together, an appropriate promoter or promoter/enhance combination, and a gene of interest, comprise an expression cassette.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202 and U.S. Pat. No. 5,928,906, each incorporated herein by reference). Such promoters may be used to drive β-galactosidase expression for use as a reporter gene. Other reporters including, but not limited to, luciferase and green flourescent protein (GFP) may also be used. Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (2001), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Various promoters may be utilized in the context of the present invention to regulate the expression of a delivered p53 gene. Of particular interest are tissue-specific promoters or elements, which permit tissue selective or preferential expression of p53. An exemplified promoter that is active in vascular smooth muscle cells is the SM-22α promoter. Appropriate sequences can be found in U.S. Pat. Nos. 5,837,534 and 6,015,711, incorporated herein by reference. Other promoters suitable for use in the present invention include those from the smooth muscle calponin gene, the smooth muscle myosin heavy chain gene, human P2x1 gene, the large isoform of smoothelin gene, flt-1 gene, endothelin 1 gene, α- and β-tropomyosin genes, α integrin gene, caldesmon gene, PCNA gene and cyclin D and E genes.

2. Initiation Signals

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” and contiguous to the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

3. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (Chandler et al., 1997).

4. Polyadenylation Signals

One may include a polyadenylation signal in the expression construct to effect proper polyadenylation of the transcript. The nature of the polyadenylation signal is not believed to be crucial to the successful practice of the invention, and/or any such sequence may be employed. Specific embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

5. Termination Signals

The vectors or constructs of the present invention may comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

6. Origins of Replication

In order to propagate a vector in a host cell, it may contain one or more origin of replication sites (often termed “ori”), which is a specific nucleic acid sequence at which replication is initiated. Alternatively an autonomously replicating sequence (ARS) can be employed if the host cell is yeast.

7. Selectable and Screenable Markers

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker. Examples of selectable and screenable markers are well known to one of skill in the art.

8. IRES

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (U.S. Pat. Nos. 5,925,565 and 5,935,819; PCT/US99/05781).

IV. Gene Transfer

There are a number of ways in which p53 expression vectors may introduced into cells. In certain embodiments of the invention, the expression construct comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene vectors were DNA viruses including the papovaviruses (simian virus 40, bovine papilloma virus, and polyoma) (Ridgeway, 1988; Baichwal and Sugden, 1986) and adenoviruses (Ridgeway, 1988; Baichwal and Sugden, 1986).

1. Adenovirus Expression Vectors

The expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus et al., 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage.

In one system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the preferred helper cell line is 293.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus SEROtype 5 of subgroup C is the preferred starting material in order to obtain the conditional replication-defective adenovirus vector for use in the present invention. This is because Adenovirus SEROtype 5 is a human adenovirus about which a great deal of biochemical and genetic information is known, and it has historically been used for most constructions employing adenovirus as a vector.

Adenovirus vectors have been used in eukaryotic gene expression (Levrero et al., 1991; Gomez-Foix et al., 1992) and vaccine development (Grunhaus et al., 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

2. Retrovirus Expression Vectors

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

3. Herpesvirus Expression Vectors

Herpes simplex virus (HSV) has generated considerable interest in treating nervous system disorders due to its tropism for neuronal cells, but this vector also can be exploited for other tissues given its wide host range. Another factor that makes HSV an attractive vector is the size and organization of the genome. Because HSV is large, incorporation of multiple genes or expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations.

HSV also is relatively easy to manipulate and can be grown to high titers. Thus, delivery is less of a problem, both in terms of volumes needed to attain sufficient MOI and in a lessened need for repeat dosings. For a review of HSV as a gene therapy vector, see Glorioso et al. (1995).

HSV, designated with subtypes 1 and 2, are enveloped viruses that are among the most common infectious agents encountered by humans, infecting millions of human subjects worldwide. The large, complex, double-stranded DNA genome encodes for dozens of different gene products, some of which derive from spliced transcripts. In addition to virion and envelope structural components, the virus encodes numerous other proteins including a protease, a ribonucleotides reductase, a DNA polymerase, a ssDNA binding protein, a helicase/primase, a DNA dependent ATPase, a dUTPase and others.

HSV genes form several groups whose expression is coordinately regulated and sequentially ordered in a cascade fashion (Honess and Roizman, 1974; Honess and Roizman 1975; Roizman and Sears, 1995). The expression of α genes, the first set of genes to be expressed after infection, is enhanced by the virion protein number 16, or α-transinducing factor (Post et al., 1981; Batterson and Roizman, 1983). The expression of β genes requires functional a gene products, most notably ICP4, which is encoded by the α4 gene (DeLuca et al., 1985). y genes, a heterogeneous group of genes encoding largely virion structural proteins, require the onset of viral DNA synthesis for optimal expression (Holland et al., 1980).

In line with the complexity of the genome, the life cycle of HSV is quite involved. In addition to the lytic cycle, which results in synthesis of virus particles and, eventually, cell death, the virus has the capability to enter a latent state in which the genome is maintained in neural ganglia until some as of yet undefined signal triggers a recurrence of the lytic cycle. Avirulent variants of HSV have been developed and are readily available for use in gene therapy contexts (U.S. Pat. No. 5,672,344).

4. Adeno-Associated Virus Expression Vectors

Recently, adeno-associated virus (AAV) has emerged as a potential alternative to the more commonly used retroviral and adenoviral vectors. While studies with retroviral and adenoviral mediated gene transfer raise concerns over potential oncogenic properties of the former, and immunogenic problems associated with the latter, AAV has not been associated with any such pathological indications.

In addition, AAV possesses several unique features that make it more desirable than the other vectors. Unlike retroviruses, AAV can infect non-dividing cells; wild-type AAV has been characterized by integration, in a site-specific manner, into chromosome 19 of human cells (Kotin and Berns, 1989; Kotin et al., 1990; Kotin et al., 1991; Samulski et al., 1991); and AAV also possesses anti-oncogenic properties (Ostrove et al., 1981; Berns and Giraud, 1996). Recombinant AAV genomes are constructed by molecularly cloning DNA sequences of interest between the AAV ITRs, eliminating the entire coding sequences of the wild-type AAV genome. The AAV vectors thus produced lack any of the coding sequences of wild-type AAV, yet retain the property of stable chromosomal integration and expression of the recombinant genes upon transduction both in vitro and in vivo (Berns, 1990; Berns and Bohensky, 1987; Kearns et al., 1996; Ponnazhagan et al., 1997a). Until recently, AAV was believed to infect almost all cell types, and even cross species barriers. However, it now has been determined that AAV infection is receptor-mediated (Ponnazhagan et al., 1996; Mizukami et al., 1996).

AAV utilizes a linear, single-stranded DNA of about 4700 base pairs. Inverted terminal repeats flank the genome. Two genes are present within the genome, giving rise to a number of distinct gene products. The first, the cap gene, produces three different virion proteins (VP), designated VP-1, VP-2 and VP-3. The second, the rep gene, encodes four non-structural proteins (NS). One or more of these rep gene products is responsible for transactivating AAV transcription. The sequence of AAV is provided in U.S. Pat. No. 5,252,479 (entire text of which is specifically incorporated herein by reference).

The three promoters in AAV are designated by their location, in map units, in the genome. These are, from left to right, p5, p19 and p40. Transcription gives rise to six transcripts, two initiated at each of three promoters, with one of each pair being spliced. The splice site, derived from map units 42-46, is the same for each transcript. The four non-structural proteins apparently are derived from the longer of the transcripts, and three virion proteins all arise from the smallest transcript.

AAV is not associated with any pathologic state in humans. Interestingly, for efficient replication, AAV requires “helping” functions from viruses such as herpes simplex virus I and II, cytomegalovirus, pseudorabies virus and, of course, adenovirus. The best characterized of the helpers is adenovirus, and many “early” functions for this virus have been shown to assist with AAV replication. Low level expression of AAV rep proteins is believed to hold AAV structural expression in check, and helper virus infection is thought to remove this block.

5. Vaccinia Virus Expression Vectors

Vaccinia virus vectors have been used extensively because of the ease of their construction, relatively high levels of expression obtained, wide host range and large capacity for carrying DNA. Vaccinia contains a linear, double-stranded DNA genome of about 186 kb that exhibits a marked “A-T” preference. Inverted terminal repeats of about 10.5 kb flank the genome. The majority of essential genes appear to map within the central region, which is most highly conserved among poxyiruses. Estimated open reading frames in vaccinia virus number from 150 to 200. Although both strands are coding, extensive overlap of reading frames is not common.

At least 25 kb can be inserted into the vaccinia virus genome (Smith and Moss, 1983). Prototypical vaccinia vectors contain transgenes inserted into the viral thymidine kinase gene via homologous recombination. Vectors are selected on the basis of a tk-phenotype. Inclusion of the untranslated leader sequence of encephalomyocarditis virus, the level of expression is higher than that of conventional vectors, with the transgenes accumulating at 10% or more of the infected cell's protein in 24 h (Elroy-Stein et al., 1989).

6. p53 Gene Delivery Using Modified Viruses

A p53-encoding nucleic acid may be housed within a viral vector that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

7. Non-Viral Methods for Transfer of Expression Constructs

In certain embodiments, a plasmid vector is contemplated for use to transform VSMC. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the host cell for the expression of p53.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Several non-viral methods for the transfer of expression constructs into mammalian cells also are contemplated by the present invention. In one embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest also may be transferred in a similar manner in vivo and express the gene product.

In a further embodiment of the invention, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al. (1980) demonstrated the feasibility of liposome-mediated delivery and expression of foreign DNA in cultured chick embryo, HeLa and hepatoma cells. Nicolau et al. (1987) accomplished successful liposome-mediated gene transfer in rats after intravenous injection. Of particular interest are the methods and compositions disclosed in PCT/US00/14350, incorporated by reference herein.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato, et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993). Receptor-mediated gene targeting vehicles generally consist of two components: a cell receptor-specific ligand and a DNA-binding agent. In other embodiments, the delivery vehicle may comprise a ligand and a liposome.

V. Vascular Insults

The present invention finds particular use in the prevention and treatment of vascular stenosis arising from arterial or venous trauma. Unfortunately, such traumas often arise from the placement of intravascular devices designed to treat other pathologic states. Such devices include catheters, such as those used for the delivery of diagnostic or therapeutic agents, or for angioplasty (e.g., “balloon catheterization”), stents or grafts.

In addition, the development of atherosclerotic plaques is another important condition in which vascular stenosis occurs. The conditions are described in depth below.

1. Catheters

A variety of catheters for varying use are commercially available. The dispatch catheter (infusion-perfusion catheter; Boston Scientific, Maple Grove, Minn.), “local collar-reservoir delivery device” and “extravascular delivery collar” (Eurogene), Wolinsky (perforated balloon) catheter, infiltrator catheter (InterVentional Technologies, San Diego, Calif.), PTCA balloon catheter, crescendo catheter (Cordis, Warren, N.J.), microporous pressure driven balloon catheter, infusasleeve catheter (LocalMed, Palo Alto, Calif.), remedy balloon or “channel balloon” (Boston Scientific), angioplasty balloon coated with hydrogel polymer (Slider with Hyrdoplus, Mansfield Boston Scientific), and double balloon catheters are all examples.

A wide variety of vascular catheters are found in the following U.S. Patents: 6,290,689; 6,234,971; 6,217,566; 6,179,825; 6,152,912; 5,951,539; 5,782,809; 5,776,096; 5,771,895; 5,755,707; 5,676,670; 5,626,602; 5,569,200; 5,554,118; 5,531,719; 5,507,766; 5,472,418; 5,405,380; 5,395,335; 5,358,493; 5,256,146; 5,176,661; RE33,925; 5,041,089; 4,950,238; 4,932,959; 4,808,158; 4,439,185; 4,411,055; and 4,323,071.

2. Stents

Stents are devices used to delivery endovascular devices, such as grafts. However, they are also used in a more permanent basis for reinforcing recently reopened veins and arteries. This procedure typically uses a wire mesh tube (the stent) to prop open an artery that's recently been cleared using angioplasty. The stent is collapsed to a small diameter, placed over an angioplasty balloon catheter and moved into the area of the blockage. When the balloon is inflated, the stent expands, locks in place and forms a scaffold to hold the artery open. The stent stays in the artery permanently, holds it open, improves blood flow to the heart muscle and relieves symptoms. Self-opening stents are another class of stents that may be used with balloon angioplasty.

The following is a list contains examples of various stents: U.S. Pat. Nos. 6,293,968; 6,267,777; 6,241,746; 6,187,035; 6,165,209; 6,053,940; 5,997,573; 5,925,061; 5,769,883; 5,741,274; 5,609,626; 5,551,954; 5,500,013; 5,466,242; 5,464,450; 5,443,498; 5,370;683; 5,314,472; 5,059,211.

3. Grafts

A common treatment of aneurysms or other vascular defects is to place a device within the lumen of the weakened blood vessel. The basic concept of a transluminal placement of an endovascular prosthesis for decreasing risk associated with the surgical repair of aortic aneurysms was first experimentally investigated by Balko (1986). Since then, several investigators have studied the feasibility of different endovascular devices. For example Lazarus (U.S. Pat. No. 5,669,936) discloses a graft system having a capsule catheter that is deployed after femoral arteriotomy. More recently, a catheter-based system for the delivery of grafts for repair of aortic aneurysms was disclosed by Taheri et al (U.S. Pat. Nos. 5,713,917 and 5,591,195). The system includes a single stage graft comprised of two nitinol springs. The two nitinol springs are in physical communication with each other via a nitinol connecting bar and are embedded in graft material at each end and covered completely by material so as to prevent direct exposure to bodily fluids or tissues. The graft is deployed by using an elongated sheath introducer having an axially extending sheath passage for receiving the graft and maintaining it in a compressed condition. A flexible push rod around the insertion catheter and within the sheath passage is used to push the graft out of the sheath during deployment.

The material is chosen so that the graft is capable of substantially deforming to conform to an interior surface of a blood vessel. Suitable synthetic materials include, but are not limited to, woven polyester, polytetrafluoroethylene (PTFE), Dacron®, microporous urethane, nylon, and lycra. A preferred fabric material is thin polyester. Graft material that is minimally porous, or even non-porous may be utilized. For example, a material such as PeCap® polyester (commercially available from Tetko, Inc., Briarcliff Manor, N.Y.) having a pore size of 5 micron, a fabric thickness of 65 micron, and an open area of 2% may be used.

Various vascular grafts are disclosed in U.S. Pat. Nos. 6,267,834; 6,253,768; 6,210,422; 6,168,620; 6,162,247; 6,120,532; 6,102,918, 6,036,724; 6,019,788; 6,015,422; 5,989,287; 5,919,233; 5,910,168; 5,891,195; 5,880,090; 5,871,536; 5,866,217; 5,851,230; 5,849,036; 5,840,240; 5,827,327; 5,824,047; 5,800,512; 5,747,128; 5,176,395; 5,700,287; 5,693,745; 5,653,745; 5,634,941; 5,628,786; 5,609,624; 5,607,464; 5,509,931; 5,496,364; 5,453,084; 5,413,598; 5,385,580; 5,282,848; 5,282,846; 5,246,452; 5,197,977; 5,197,976; 5,192,310; 5,156,619; 5,127,919; 5,123,917; 5,104,402; 5,024,671; 4,997,440; 4,969,896; 4,955,899; 4909,979; 4,892,539; 4,816,028; 4,695,280; 4,647,416; 4,601,718; 4,550,447; 4,487,567; 4,459,252; 4,441,215; 4,355,426; 4,047,252; 3,945,052.

4. Arteriosclerosis/Atherosclerosis

Arteriosclerosis comprises a group of disorders that causes thickening and loss of elasticity of artery walls. The most common form of arteriosclerosis is atherosclerosis, which is characterized by an inner layer of thickness in the arterial wall, causing narrowing of the channel and impairing blood flow. The narrowing is due to the formation of plaques in the artery lining, consisting of low density lipoproteins, decaying muscle cells, fibrous tissue, clumps of blood platelets, and cholesterol. They tend to form in regions of turbulent blood flow and are most common in individuals with high cholesterol levels. Persistence of the plaques may lead to formation of emboli.

Atherosclerosis is a major cause of coronary heart disease, and can contribute to other serious illnesses by reducing blood flow to other organs such as kidneys, legs and intestines. Modification of risk factors can markedly reduce the incidence of atherosclerosis, or at least delay its onset. Unfortunately, many individuals fail to avoid risk factors until such time as symptoms appear, but by this time, the damage has already been done. Anticoagulants and vasodilators can reduce the effects of the disease, and angioplasty can open up the restricted arteries. In addition, severe cases may be dealt with by arterial bypass. None of these options is entirely satisfactory.

Thus, in accordance with the present invention, the inventors propose to intervene prophylactically by treating very early stage atherosclerotic lesions, as well as patients with later stage disease. In order to achieve this goal, the p53 expression vectors of the present invention will be administered to individuals that suffer from one or more of the predominant risk factors for atherosclerosis. Such risk factors include smoking, hypertension, elevated cholesterol levels and obesity. Treatment may be systemic or directed to particular regions where atherosclerotic plaques tend to form, such as coronary arteries and peripheral arteries.

5. Transplantation

Another potential use for the present invention is in the context of organ transplant or ex vivo graft. Due to various insults on the vasculature of transplanted organs, subsequent stenosis may severely restrict the perfusion of the organ, thereby reducing its function and, perhaps, its very viability. Thus, the delivery of p53 to the vasculature of a transplanted organ may prevent this loss of function.

6. Peripheral Vascular (Arterial) Disease Applications

Peripheral arterial disease (PAD) becomes increasingly common with age. An estimated 12-17% of the population over age 50 have PAD. Increased mortality has been well documented in patients with PAD, a disease that is strongly associated with coronary artery disease and that shares many of the same risk factors. Although only a small proportion of individuals with PAD and intermittent claudication develop skin breakdown or limb loss, pain and associated disability often restrict ambulation and the overall quality of life. Persons at increased risk for PAD include cigarette smokers and persons with diabetes mellitus or hypertension. Diabetic PAD is responsible for about 50% of all amputations.

The present invention also contemplates treatment of PVD and PAD with p53 vectors of the present invention. There is evidence that a history of intermittent claudication and the palpation of peripheral pulses are unreliable techniques for the detection of PAD. Greater accuracy has been achieved with noninvasive testing using Doppler ankle-arm pressure ratios, measurement of reactive hyperemia after exercise, pulse reappearance time, ultrasound duplex scanning, and plethysmography.

VI. Combination Therapy

1. General

To inhibit proliferation or migration of VSMC, using the methods and compositions of the present invention, one may use a p53 therapy in conjunction with at least one other agent. These two compositions would be provided in a combined amount effective to inhibit proliferation or migration of the treated cells. This process may involve contacting the cells with the p53 expression construct and the other agents at the same time. This may be achieved by contacting the cell with a single composition or pharmacological formulation that includes both the p53 construct and the agents, or by contacting the cell with two distinct compositions or formulations, at the same time, wherein one composition includes the expression construct and the other includes the agent.

Alternatively, the p53 gene therapy treatment may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one would contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is conceivable that more than one administration of either the p53 construct and/or the other agent will be desired. Various combinations may be employed, where the p53 expression construct is “A” and the other agent is “B”, as exemplified below:

| | | | | | | | |
|---|---|---|---|---|---|---|---|
| A/B/A | B/A/B | B/B/A | A/A/B | B/A/A | A/B/B | B/B/B/A | B/B/A/B |
| A/A/B/B | A/B/A/B | A/B/B/A | B/B/A/A | B/A/B/A | B/A/A/B | B/B/B/A | |
| A/A/A/B | B/A/A/A | A/B/A/A | A/A/B/A | A/B/B/B | B/A/B/B | B/B/A/B | |

Other combinations are contemplated. Again, to achieve cell inhibition, both agents are delivered to a cell in a combined amount effective to inhibit vascular smooth muscle cell proliferation and/or migration.

2. Genes

The following is a list of genes that may be used, advantageously, in combination with p53 gene therapy.

(a) Tumor Suppressors

i. PTEN

PTEN, also known as MMAC-1 (mutated in multiple advanced cancers) and TEP-1 (TGF-beta regulated and epithelial-cell-enriched phosphatase), was reported by three groups (Li et al., 1997; Steck et al., 1997; Li & Sun, 1997). Structural comparisons of the 55 kDa protein encoded by PTEN by Myers et al. (1997) implied its function as a phosphatase, and shortly thereafter PTEN was recognized as a dual-specificity phosphatase, having ability to recognize proteins phosphorylated both on tyrosine and serine/threonine residues (Myers et al., 1997; Tonks & Myers, 1999). PTEN has both protein phosphatase and lipid phosphatase (specifically, PIP-3) activity (Maehama & Dixon, 1998). Its lipid phosphatase activity is much more active than its protein phosphatase activity (Maehama & Dixon, 1998) and is responsible for the PTEN tumor suppressor activity, as mutations which inactivate the lipid phosphatase activity but do not affect the protein phosphatase activity still inactivate the tumor suppressor effects (Myers et al., 1998; Furnari et al., 1998).

In tumors, mutations in PTEN are often localized around the phosphatase domain (Myers et al., 1998). Deficiency in the lipid phosphatase ability of PTEN correlates with its loss of tumor suppressor activity. PTEN has homology to tensin, an actin-binding protein co-localizing with focal adhesion complex (Ramaswamy et al., 1999). The capacity of PTEN to regulate cell cycle and cell survival functions resembles that of the tumor suppressor gene, p53, although its actions occur through entirely different pathways. A key downstream target of PTEN is the cell-survival factor Akt, which suppresses apoptosis by inhibiting the activation of the caspase cascade, specifically the phosphorylation of caspase-9 (Cardone et al., 1998) and possibly by other mechanisms (Jung et al., 2000).

PTEN acts to dephosphorylate and thereby reduce phosphoinositol precursors of Akt, directly opposing the role of P13 kinase. PTEN serves to limit PIP3 and PIP2, thereby limiting the formation of activated Akt and other protein kinases. As shown by Jung et al. (2000), cell proliferation is mediated, at least partially, by the phosphatidylinositol-3-OH kinase (P13K) pathway (Jung et al., 2000). PTEN has also been shown to bind to focal adhesion kinase (FAK) (Tamura et al., 1998) and inhibits cell adhesion and migration. By dephosphorylating D3 residues on PIP3 and PIP2, PTEN functions to oppose the P13K/Akt pathway, thereby counteracting cell survival mechanisms stabilized by P13K/Akt.

The coding sequence for PTEN can be obtained through GenBank Accession No. HSU93051, and the genomic sequences is at No. AF067844. Also, PCT/GB96/02588, filed Oct. 22, 1996, which describes various aspects of PTEN, is specifically incorporated herein by reference.

ii. p16

The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit p16 NK4. The p16^{INK4} has been biochemically characterized as a protein that specifically binds to and inhibits CDK4, and thus may regulate Rb phosphorylation (Serrano et al., 1993; Serrano et al., 1995). Since the p16^{INK}⁴ protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a class of CDK-inhibitory proteins that also includes p15^{INK4B}, p21 WAF1, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Okamoto et al., 1994; Nobori et al., 1995; Orlow et al., 1994; Arap et al., 1995). However, it was later shown that while the p16 gene was intact in many primary tumors, there were other mechanisms that prevented p16 protein expression in a large percentage of some tumor types. p 16 promoter hypermethylation is one of these mechanisms (Merlo et al., 1995; Herman et al., 1995; Gonzalez-Zulueta, 1995). Restoration of wild-type p16^{INK4} function by transfection with a plasmid expression vector reduced colony formation by some human cancer cell lines (Okamoto, 1994; Arap, 1995). Delivery of p16 with adenovirus vectors inhibits proliferation of some human cancer lines and reduces the growth of human tumor xenografts.

iii. C-CAM

C-CAM is expressed in virtually all epithelial cells (Odin and Obrink, 1987). C-CAM, with an apparent molecular weight of 105 kD, was originally isolated from the plasma membrane of the rat hepatocyte by its reaction with specific antibodies that neutralize cell aggregation (Obrink, 1991). Recent studies indicate that, structurally, C-CAM belongs to the immunoglobulin (Ig) superfamily and its sequence is highly homologous to carcinoembryonic antigen (CEA) (Lin and Guidotti, 1989). Using a baculovirus expression system, Cheung et al. (1993) demonstrated that the first Ig domain of C-CAM is critical for cell adhesive activity.

Cell adhesion molecules, or CAM's are known to be involved in a complex network of molecular interactions that regulate organ development and cell differentiation (Edelman, 1985). Recent data indicate that aberrant expression of CAM's maybe involved in the tumorigenesis of several neoplasms; for example, decreased expression of E-cadherin, which is predominantly expressed in epithelial cells, is associated with the progression of several kinds of neoplasms (Edelman and Crossin, 1991; Frixen et al., 1991; Bussemakers et al., 1992; Matsura et al., 1992; Umbas et al., 1992). Also, Giancotti and Ruoslahti (1990) demonstrated that increasing expression of α₅β₁ integrin by gene transfer can reduce tumorigenicity of Chinese hamster ovary cells in vivo. C-CAM now has been shown to suppress tumor growth in vitro and in vivo.

iv. Other Tumor Suppressors

Other tumor suppressors that may be employed according to the present invention include p21, p15, BRCA1, BRCA2, IRF-1, RB, APC, DCC, NF-1, NF-2, WT-1, MEN-I, MDA7, MEN-II, zac1, p73, VHL, FCC, MCC, DBCCR1, DCP4 and p57.

(b) Inducers of Apoptosis

Inducers of apoptosis, such as Bax, Bak, Bcl-X₅, Bad, Bim, Bik, Bid, Harakiri, Ad E1B, Bad, ICE-CED3 proteases, TRAIL, SARP-2 and apoptin, similarly could find use as secondary gene therapies according to the present invention.

(c) Cell Cycle Regulatory Proteins

Cell cycle regulators provide possible advantages, when combined with the p53 gene therapy of the present invention. Such cell cycle regulators include E2F-1, E2F-2, E2F-3, p107, p130 and E2F-4. Other cell cycle regulators include anti-angiogenic proteins, such as soluble Flt1 (dominant negative soluble VEGF receptor), soluble Wnt receptors, soluble Tie2/Tek receptor, soluble hemopexin domain of matrix metalloprotease 2 and soluble receptors of other angiogenic cytokines (e.g., VEGFR1/KDR, VEGFR3/Flt4, both VEGF receptors).

(d) Toxins

Various toxins are also contemplated to be useful in conjunction with p53 expression vectors of the present invention and include bacterial toxins such as ricin A-chain (Burbage, 1997), diphtheria toxin A (Massuda et al., 1997; Lidor, 1997), pertussis toxin A subunit, E. coli enterotoxin toxin A subunit, cholera toxin A subunit and Pseudomonas toxin c-terminal.

(e) Enzymes

i. COX-1

Experimental data have indicated that the constitutive cyclooxygenase (COX-1, or PGH synthase-1) is the key rate-limiting step in controlling the extent of prostacyclin synthesis. Overexpression of COX-1 in a cultured human endothelial cell line by retrovirus-mediated transfer of COX-1 cDNA is accompanied by a sustained increase in prostacyclin synthesis. This in vitro gene transfer result suggests that local production of prostacyclin to the vascular injury sites is augmented by transfer of COX-1. Prostacyclin is a key vasoprotective molecule which is synthesized in vascular cells, and acts as an autacoid in inhibiting platelet aggregation and maintaining smooth muscle (SMC) relaxation. Injury to the endothelium causes a reduction in prostacyclin synthesis with an attendant risk of thrombosis. Thus, in accordance with the present invention, provision of a COX-1 gene in combination with p53 gene therapy is envisioned as a particularly effective therapy.

ii. NOS

There are three forms of nitric oxide synthase—a neuronal type called nNOS, an epithelial type called eNOS, and an inducible form called iNOS. The latter is only expressed under certain conditions like immune system regulation by cytokines or pathological induction in the presence of endotoxins (bacterial lipopolysaccharide) and cytotoxins (which affect cytokine secretion). NO production is a stress response and can lead to either tissue injury because of its radical chemistry, or be cytoprotective, protecting cells from damage by destroying pathogenic microorganisms first. eNOS and iNOS are appropriate combination gene therapies in accordance with the present invention.

iii. TIMP

TIMPs (tissue inhibitors of metalloproteinase) have been studied as gene therapeutic agents in monotherapies for vascular stenosis. Studies in isolated cells have demonstrated that over expression of TIMP-1, -2 and -3 significantly inhibited VSMC migration and MMP activity. In addition, TIMP-2 over expression inhibited VSMC proliferation and TIMP-3 over expression induced apoptosis. TIMP-1, TIMP-2 and TIMP-3 all inhibited intimal thickening (George et al., 1996; 1997; 1998a; 1998b; 2000; Baker et al., 1998; Southgate et al., 1999).

iv. TFPI

Tissue Factor Pathway Inhibitor (TFPI) is an inhibitor of tissue factor-mediated coagulation that has a central role in the modem hypothesis of coagulation. The regulatory role of this inhibitor has redefined the classical extrinsic (in which TFPI is involved) and intrinsic pathways of coagulation and helps to explain why haemophiliacs bleed. Augmentation of TFPI levels may provide treatment for thrombotic disorders such as disseminated intravascular coagulation and deep vein thrombosis. Full length and truncated forms of TFPI molecules are also being considered as antithrombotic agents. The protein has three Kunitz domains; these are domains which can bind to and inhibit serine proteases. The first Kunitz domain inhibits factor VIIa in the tissue factor:VIIa complex. The second Kunitz domain inhibits factor Xa. The fact that thrombus formation is intrinsic to stenosis makes TFPI molecules important in reducing this aspect of the disease.

3. Pharmaceuticals

There already exist a number of drugs that can be used to treat various aspects of vascular disease. Certain of these find particular value in combating vascular stenosis in combination with the p53 gene therapy of the present invention.

(a) Anti-Thrombotics

In one embodiment, the present invention provides a combination therapy involving the use of a p53 expression construct and one or more anti-thrombotic agents. Anti-thrombotics inhibit thrombus (clot) formation. Aspirin is one of the most common anti-thrombotics. It inhibits cyclooxygenase, leading to suppression of thromboxane A2 and platelet aggregation. The typical dose is 80 to 325 mg/day, unless contraindicated by, e.g., peptic ulcer or GI hemorrhage.

Another class of anti-thrombotics is the statins. These are HMG-CoA reductase inhibitors. They inhibit platelet aggregation and reduce fibrinogen levels. Examples include Lovastatin (Mevacor), Pravastatin (Pravachol), Simvastatin (Ticlopidine; Ticlid), Clopidogrel (Plavix).

Yet another anti-thrombotic is Cilostazol, a potent anti-platelet agent with anti-proliferative properties. Other anti-platelets agents include dipyridamole (Persantine) and Aggrenox (dypyrimidole+aspirin).

(b) Fibrolytic Agents

Another general class of compounds that are useful in combination with p53 gene therapy is the fibrolytics. Aminocaproic acid (Amicar), Streptokinase, Anistreplase (plasminogen and streptokinase) and tissue Plasminigen Activator (tPA) are the most common.

(c) Other Agents

No pharmacologic agents have proven, in and of themselves, to be clinically effective in reducing restenosis. However, a number of agents provide useful effects that make them suitable combination drugs. Such other agents include thromboxane antagonists, platelet glycoprotein IIb/IIIa inhibitors, Abciximab (ReoPro; aspirin+heparin), Eptifibatide (Integrilin), Tirofiban (Aggrastat) and Lamifiban, anti-coagulants (Warfarin, Heparin, Hirudin, Hirulog), vasodilators, thromboxane antagonists (prostacyclins), calcium channel antagonists, ACE inhibitors, antiproliferative agents, growth factor inhibitors, and anti-inflammatory agents (Tranilast).

Currently, the most common post-stent treatments are (a) aspirin 325 mg>12 h prior, and indefinitely thereafter; plus (b) either ticlopidine at 250 mg 2 times/d or clopidogrel 75 mg/d started >72 h before procedure and continued for 2-4 w; plus (c) iv heparin 100 U/kg administered after gaining arterial access, to achieve and maintain an ACT of 200-300 s. Patients treated concomitantly with GPIIb/IIIa inhibitors should receive 70 U/kg only, to maintain an ACT of 200-300 s. Heparin should be discontinued at the end of the procedure.

Clinical trials are underway for rapamycin (Sirolimus)-coated stents, paclitaxel-coated stents, Trapidil, which suppresses PDGF, inhibits neointimal hyperplasia, decreases SMC proliferation, Probucol (Lorelco) an anti-hyperlipidemic, and Tranilast, an anti-inflammatory agent that restores nitric oxide, inhibits SMC migration and proliferation, and inhibits neointima formation in animal models. Also being examined are ultrasound and radioactive stents.

(d) Endoluminal Radiation

U.S. Pat. No. 5,899,882 discloses an apparatus and method for delivery of a treating element, such as a radiation source, through a catheter to a desired site in the endoluminal passageways of a patient, such as a coronary artery, for inhibiting the formation of scar tissue such as may occur in restenosis following balloon angioplasty. The apparatus includes an elongated flexible catheter tube having proximal and distal end portions, with a lumen extending therebetween, and a diameter sufficiently small for insertion in to a patient's intraluminal passageways. One or more treating elements, such as a capsule or pellet containing radioactive material, is positionable within the lumen and movable between the proximal and distal end portions under the force of liquid flowing through the lumen. A method for using such apparatus, including a method for using such apparatus simultaneously with a balloon angioplasty procedure, is disclosed.

The Intimal Hyperplasia Inhibition with Beta In-stent Trial (INHIBIT) demonstrated a 33-66% reduction in the rates of in-stent restenosis and MACE, and no increase in the rates of late thrombosis and edge-effect at 9 months with beta radiation therapy as compared to placebo. The Stent And Radiation Therapy (START) involved Novoste Corp.'s BetaCath system (Sr-90 source) in the treatment of in-stent restenosis, and the results were so successful that shortly thereafter the BetaCath system received FDA approval. INHIBIT is a Guidant Corp-sponsored trial that involves Guidant's investigational Galileo(™) Intravascular Radiotherapy System (P-32 source). In INHIBIT, 332 patients, who had been successfully treated with conventional therapy for in-stent restenosis, were randomly assigned to intracoronary radiation—a dose of 20 Gy at 1 mm beyond the lumen diameter—or placebo.

VII. Pharmaceutical Formulations and Delivery

Pharmaceutical compositions of the present invention comprise an effective amount of a p53 expression construct and optionally additional agent(s) dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The pharmaceutical composition may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof In certain embodiments, pharmaceutical compositions are prepared for administration by oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In terms of delivery, loco-regional administration, such as to isolated limb circulation, local administration via catheter to an affected vascular zone, systemic and even ex vivo (for grafts and transplants) is envisioned. In particular, catheter delivery of both p53 expression constructs and secondary agents is provided.

### VIII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Cell lines. The normal human foreskin fibroblast cell line MJ90 was used. Cells were grown in complete media: DMEM (GIBCO, Cat# 11965092)+10% Fetal Bovine Serum (heat inactivated, Summit Biotechnologies, Cat# RS-50-05)+200 units/ml of Penicillin and 200 μg/ml of Streptomycin (GIBCO, Cat# 15140-122). Cultures were passaged once a week at 1:10. Cells were tested to be free of Mycoplasma (Mycoplasma T.C. Kit, Gen-Probe Inc., San Diego, Calif.).

Normal human umbilical vein endothelial cells (HUVEC) were obtained from Clonetics (San Diego, Calif.). Cells were lot 8F2065, a pooled population of four human donors (Cat #CC-2519). Cells were grown in endothelial growth media (EGM) composed of endothelial basal media (EBM) supplemented with 2% fetal bovine serum (FBS), 1.2 μg/ml bovine brain extract (BBE), 10 ng/ml human recombinant epidermal growth factor (hEGF), 1 μg/ml hydrocortisone, 50 μg/ml gentamicin and 50 ng/ml Amphotericin B (all from Clonetics, Cat# CC-3124). Cultures were passaged twice a week at 1:10. Cells were tested to be free of Mycoplasma (Mycoplasma T.C. Kit, Gen-Probe Inc., San Diego, Calif.). Quiescent HUVEC cells were obtained by culturing cells to confluency and maintaining them at confluency for a period of 10 days; the majority of cells were no longer actively dividing at this point but were viable.

HUVEC used for the tritiated thymidine incorporation assays were obtained from ATCC (ATCC Cat# CRL-1730, St. Louis, Mo.). Cells were grown in CS-C Medium with Serum for Endothelial Cell Lines (Sigma, Cat# C1431) supplemented with 1% Endothelial Cell Growth Factor (Sigma Cat# E9640). Labware used to culture HUVEC was coated with Endothelial Cell Attachment Factor (Sigma Cat#E9765) prior to addition of cells. Trypsin/EDTA Solution for Endothelial Cell Cultures (Sigma Cat#T4299) and Trypsin Inhibitor Solution (Sigma Cat#T0800) were used to passage the cells.

Normal human mammary epithelial cells (HMEC) were obtained from Clonetics (San Diego, Calif.). Cells were grown in mammary epithelial basal media (MEBM) supplemented with 52 μg/ml bovine pituitary extract (BPE), 10 ng/ml human recombinant epidermal growth factor (hEGF), 5 μg/ml insulin, 500 ng/ml hydrocortisone, 50 μg/ml gentamicin and 50 ng/ml Amphotericin B (all from Clonetics, Cat # cc-3150). Normal human coronary artery smooth muscle cells (HCASMC) were obtained from Clonetics (Cat# CC-2583, San Diego, Calif.). The two lots used were OF1129 and 16445. Cells were grown in Smooth Muscle Cell Growth Media (SmGM-2) consisting of Smooth Muscle Basal Media (SmBM) supplemented with 5% fetal bovine serum (FBS), 10 ng/ml human recombinant epidermal growth factor (hEGF), 2 ng/ml human recombinant fibroblast growth factor (hFGF), 390 ng/ml dexamethasone, 50 ng/ml gentamicin, and 50 ng/ml Amphotericin B (all from Clonetics, Cat# CC-3182). Cultures were passaged twice a week at 1:10 or 1:20.

Normal human hepatocytes were obtained either from Clonetics (San Diego, Calif.) or from In Vitro Technologies, Inc. (Baltimore, Md.). Clonetics hepatocytes were cultured in Hepatocyte Maintenance Media (HMM) (Clonetics Cat # cc-3197) supplemented with 0.1 μM insulin, 0.1 μM dexamethasone, 50 μg/ml gentamicin, and 50 ng/mL Amphotericin B (all from Clonetics, Cat # cc-4192). Cells were received pre-plated at a density of 6×10⁶ cells/96-well plate on collagen coated plates. Cells were replenished daily with 100 μl HMM/well and incubated at 37° C. at 5% CO₂. In Vitro Technologies, Inc. hepatocytes were received pre-plated at a density of 5×10⁴ cells/well in a 96-well plate and were cultured in IVT Hepatocyte Culture Media. This proprietary media contained the antibiotics penicillin and streptomycin as well as the antifungal/antimycotic Fungizone. Cells were incubated at 37° C. at 5% CO₂. It should be noted that both types of hepatocytes do not actively proliferate.

Adenoviral vectors. Adenovirus infection was based on multiplicities of infection (MOIs) in viral particles per cell (vp/cell). Ad-luciferase (Ad-Luc) is a negative control adenoviral vector that encodes luciferase. Ad5CMV-p53 (INGN 201, or Ad-p53) has been described Zhang et. al. (1993) and Bacchetta et. al. (1999). Ad-Luc (Introgen, lots #697004 and lot#1) had titers of 5×10¹² μp/ml and 5.5×10¹² vp/ml, respectively. Ad5CMV-p53 (Introgen, lots #45-89PDCC11, B1739601, and L/N 201-01-1-001) had titers of 2.48×10¹² μp/ml, 6.55×10¹² vp/ml, and 1×10¹² vp/ml, respectively. Ad-βgal (Introgen, lot # B3569901) had a titer of 1×10¹² vp/ml.

Tritiated thymidine incorporation proliferation assay. Proliferating MJ90 or HMEC were plated in 96-well plates at 2,000 and 750 cells/well, respectively, and 20,000 or 7,500 cells/well in 12-well plates, respectively, and incubated at 37° C. overnight. The next day, cells in the 12-well plates were trypsinized and counted to determine cell density for infection. Cells in the 96-well plates were either mock infected or infected with adenovirus vectors at the indicated MOIs. During infection, media was aspirated from the 96-well plates and cells were infected with 50 μl/well of virus in serum-free DMEM media. Plates were incubated at 37° C. for 1 hr with shaking every 15 minutes. After one hour infection, 150 μl/well of complete media was added. Twenty-four hour later, media was aspirated and replaced with 200 μl of virus-free complete media. Cells were analyzed at various times after infection for tritiated thymidine incorporation. Briefly, ³H-thymidine was added (20 μl/well of ³H-thymidine, 5.0 Ci/mmol in DMEM at 11 μCi/ml or 100 μl/well of ³H-thymidine, 25.0 Ci/mmol in DMEM at 55 μCi/ml) to cells the day before harvest. The reaction was stopped 15-18 hours later by freezing the plates at −80° C. Cells were collected onto filter paper using a Packard Filtermate 196™ cell harvester following the manufacturer's protocol and washed in deionized water and methanol. The filters were dried and analyzed in a Matrix 9600™ Direct Beta Counter (Packard) following the manufacturer's protocol.

HUVEC were plated in 96-well plates at 2,500 cells/well and 25,000 cells/well in 12-well plates and incubated at 37° C. overnight. The next day, cells in the 12-well plate were trypsinized and counted to determine cell density for infection. Cells in the 96-well plates were either mock infected or infected with adenovirus vectors at the indicated MOIs. During infection, media was aspirated from the 96-well plates and cells were infected with 50 μl/well of virus in serum-free DMEM media. Plates were incubated at 37° C. for 1 hr with shaking every 15 minutes. After one hour infection, 150 μl/well of CS-C media was added. Twenty-four hours later, media was aspirated and replaced with either 100 μL/well of ³H-thymidine or 80 μL/well of CS-C media followed by 20 μL/well of ³H-thymidine. Cells were analyzed at 48 and 72-hours post-infection as described above.

Cell counting and viability (trypan blue exclusion) assay. HUVEC cells were plated in 12-well plates at 5×10⁴ cells/well and incubated at 37° C. overnight. Cells were counted the next day to determine the cell density for infection. During infection, media was aspirated from the 12-well plates and cells were infected with 1 ml/well of virus in EGM. Plates were incubated at 37° C. for 1.5 hr with shaking every 15 minutes after which the virus was diluted with the addition of 3 ml EGM/well. Cells were incubated overnight before media was aspirated and replaced with 2 ml EGM. On day 3 post infection, all cells were trypsinized and counted by a trypan blue (Sigma, Cat# T-8154) exclusion method to determine cell density. Briefly, media was aspirated and the cells washed with 0.5 ml/well of Hank's Balanced Salt Solution (HBSS, Clonetics Cat#CC-5022) which was then aspirated. Cells were trypsinized by the addition of 200 μl trypsin (Clonetics Cat#CC-5012)/well until they had detached after which the trypsin was neutralized by addition of 200 μl/well of trypsin neutralization solution (Clonetics Cat#CC-5002). Cell suspensions were mixed and 360 μl trypsinized cells were added to 40 μl trypan blue. Viable cells, which had excluded the dye, were counted with a hemacytometer. Infections and trypan blue counts were carried out in triplicate.

Quiescent HUVEC cells were plated into 6-well plates and grown at 37° C. for 10 days until they were confluent. Media was aspirated and replaced on days 3 and 7 after plating. The next day, 3 wells were trypsinized and counted to determine cell density. From the calculated cell density cells were mock infected or infected with adenoviruses at various MOIs with 1 ml virus/well in EGM. Cells were infected for 3.5 hours at 37° C. after which the adenoviruses were diluted by addition of 4 ml EGM/well followed by a 24 hour incubation at 37° C. Following incubation the media was aspirated and replaced with 2 ml EGM/well. On day 2 post infection the cells in half the wells were trypsinized and counted by trypan blue (Sigma, Cat#T-8154) exclusion method to determine cell density. Note that this experiment was done concurrently with the apoptosis assay on quiescent HUVEC cells described below. The cells in the remaining half of the wells were used in that assay.

CASMC cells were plated in 12-well plates in SmGM-2 media with low serum (0.5% FBS versus 5% FBS). Cells were cultured in low serum SmGM-2 for 7 days in order to trigger quiescence. CASMC were kept sub-confluent to prevent contact inhibition. Three wells of each lot were then trypsinized and counted to determine cell density for adenoviral infection. The low serum SmGM-2 was aspirated and cells mock infected or infected with 0.5 ml adenovirus/well in regular SmGM-2 for 12 hours at 37° C. The samples were diluted with 1 ml/well of SmGM-2 and incubated 24 hours at 37° C. After incubation the media was aspirated and the cells replenished with 1 ml SmGM-2/well. On day 3 post infection the cells were trypsinized and counted by a trypan blue (Sigma, Cat# T-8154) exclusion assay as described previously.

Apoptosis assay. HUVEC and HMEC cells were plated in 6-well plates at 2×10⁵ cells/well and 3.5×10⁴ cells/well, respectively, and incubated at 37° C. for 72 or 24 hours, respectively. Cells were also plated into 12-well plates at 1×10⁵ cells/well for HUVEC and 1.75×10⁴ cells/well for HMEC and incubated for the same lengths of time. The next day, cells in the 12-well plate were trypsinized and counted to determine cell density. Cells in the 6-well plates were then either mock infected or infected with adenovirus vectors at indicated MOIs. During infection, media was aspirated from the 6-well plates and cells were infected with 1 ml/well of virus in EGM or MEGM. Plates were incubated at 37° C. for 2 hours then the viruses were diluted with 3 ml of EGM or MEGM and incubated overnight. Following incubation media was aspirated and replaced with 2 ml of EGM for HUVEC or 3 ml of MEGM for HMEC. At day 3 post infection, cells remaining adherent were trypsinized and combined with the floating cells harvested from the culture media. These cell pellets were then washed with PBS and analyzed for apoptosis by Annexin V and propidium iodide staining using Clontech's ApoAlert AnnexinV-FITC apoptosis kit (Cat#K2025-2) according to manufacturer's procedure. FACS (fluorescence-activated cell sorting) analysis of all the samples was performed. FACS sorts cells into 4 categories; viable (Quadrant III), necrotic (Quadrant I), late apoptotic/necrotic (Quadrant II), and early necrotic (Quadrant IV). In plots of non-viable cells, typically only apoptotic cells (Quadrants II and IV) are used. In some experiments the level of necrotic cells (Quadrant I) was significant and was included in the category of non-viable cells.

HUVEC were also analyzed looking at only the attached cells. Cells were plated in 6-well plates at 1×10⁵ cells/well and incubated at 37° C. for 24 hours. The next day, cells from one well were counted to determine cell density. Media was aspirated from the 6-well plates and cells were either mock infected or infected with adenovirus vectors at the desired MOIs with 1 ml virus/well in EGM for 2 hours at 37° C. The viruses were then diluted with 3 ml EGM/well and incubated overnight at 37° C. Following incubation the media was aspirated and replenished with 2 ml EGM/well. Cells were assayed at day 3 post infection as described above but without the addition of floating cells present in the culture media, which was aspirated instead.

Quiescent HUVEC were also analyzed. Cells were plated, cultured, and infected as described above for the quiescent HUVEC trypan blue assay. On day 2 post-infection the remaining wells were trypsinized and combined with the floating cells harvested from the culture media. These cell pellets were then washed with PBS and analyzed for apoptosis by Annexin V and propidium iodide staining as described above.

CASMC were plated into 12-well plates in low serum SmGM-2 and incubated for 96 hours at 37° C. Three wells of each lot were then trypsinized and counted to determine the cell density for adenoviral infection. Cells were either mock infected or infected at various MOIs with 0.5 ml adenovirus/well for 4.5 hours at 37° C. in regular (5% FBS) SmGM-2. Following incubation the adenovirus was diluted by addition of 1 ml/well of regular SmGM-2 then incubated 24 hours at 37° C. After incubation the media was aspirated and the cells replenished with 1 ml/well of SmGM-2. On day 3 post infection the media was removed and CASMC were washed with 0.5 ml/well of HBSS which was then aspirated. Cells were assayed for apoptosis as described above.

Transduction efficiency determinations. HUVEC and HMEC were plated in 12-well plates at 8.5×10⁴ cells/well and 4.5×10⁴ cells/well, respectively. Cells were incubated at 37° C. for 48 or 24 hours, respectively, after which 3 wells of a 12-well plate were trypsinized and counted to determine the cell density for adenoviral infection. Cells were infected for 5 hours (HUVEC) or 1 hour (HMEC) with 1 ml/well of Ad-βgal adenovirus in EGM or MEGM at varying MOIs at 37° C. after which the adenovirus was diluted by addition of 3 ml/well of EGM or MEGM. Cells were incubated at 37° C. for 24 hours. After incubation media was aspirated and 2 ml/well of EGM or MEGM was added. On day 2 post infection cells were assayed for βgal expression. Briefly, media was aspirated and the cells were washed with PBS. Cells were then fixed by a 5-10 min incubation at 37° C. in 1% glutaraldehyde in PBS. After washing twice with PBS to remove chelators 2 ml/well of X-gal solution was added (8.4 mM KCl, 0.84 M NaPO₄ (pH 7.5), 1 mM MgCl₂, 3 mM K4[Fe(CN)₆], 3 mM K₃[Fe(CN)₆], 0.1% Triton X-100, 0.05% (w/v) X-Gal in DMSO) and the cells were incubated overnight at 37° C. for HMEC or 4 hours at 37° C. for HUVEC. The cells were photographed and transduction efficiencies were estimated.

CASMC were plated in 6-well plates at 8.4×10⁴ cells/well and 4×10⁴ cells/well and in 12-well plates at 4.2×10⁴ cells/well and 2×10⁴ cells/well for lots 16445 and OF1129, respectively. Cells were incubated for 96 hours at 37° C. after which the cells in the 12-well plates were counted to determine cell density for adenoviral infection. Cells were infected with 0.5 ml/well of Ad-βgal adenovirus at varying MOIs at 37° C. for 3 hours. Afterwards, the Ad-βgal was diluted with 3 ml SmGM-2/well and incubated at 37° C. for 24 hours. After incubation media was aspirated and 3 ml/well of fresh SmGM-2 was added. On day 2 post-infection cells were assayed for βgal expression as described previously.

### Example 2

### Results

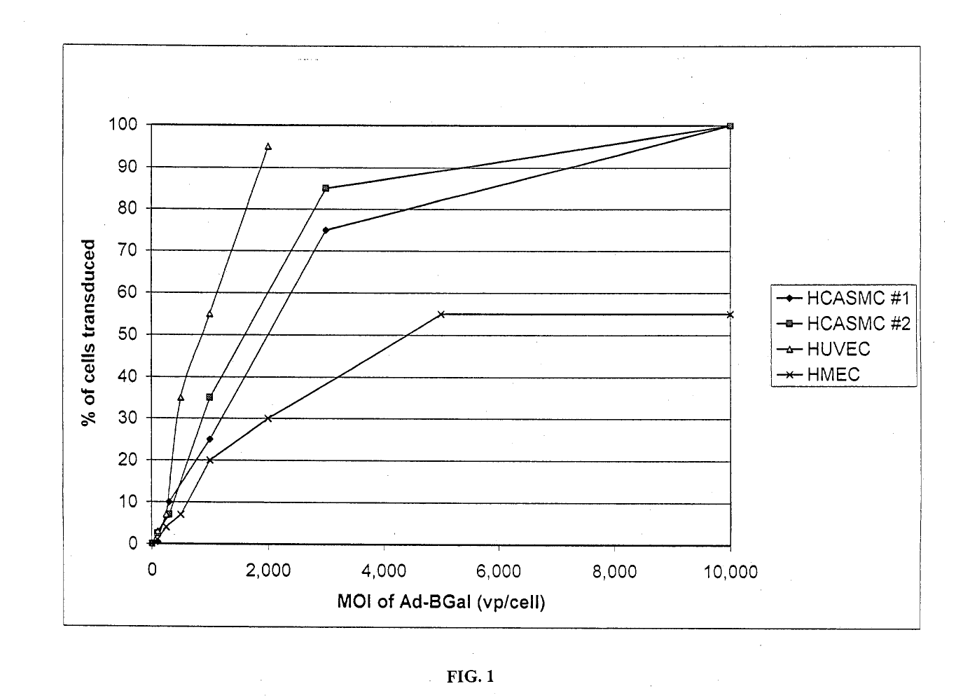

Transduction Efficiency of Normal Human Cells. The transduction efficiency of HUVEC, HMEC, and HCASMC was determined by infecting the cells with Ad-βGal, staining for βGal enzymatic activity, and visually estimating the percentage of blue (βGal positive) cells (FIG. 1). TDE50 is the MOI of Ad-βGal required for 50% blue cells. HUVEC had the lowest TDE50, at 1,000 vp/cell, followed by HCASMC at 2,000 vp/cell. HMEC were fairly resistant to transduction, with a TDE50 of 4,000 vp/cell; even at an MOI of 10,000 vp/cell, only about 55% of cells were blue.

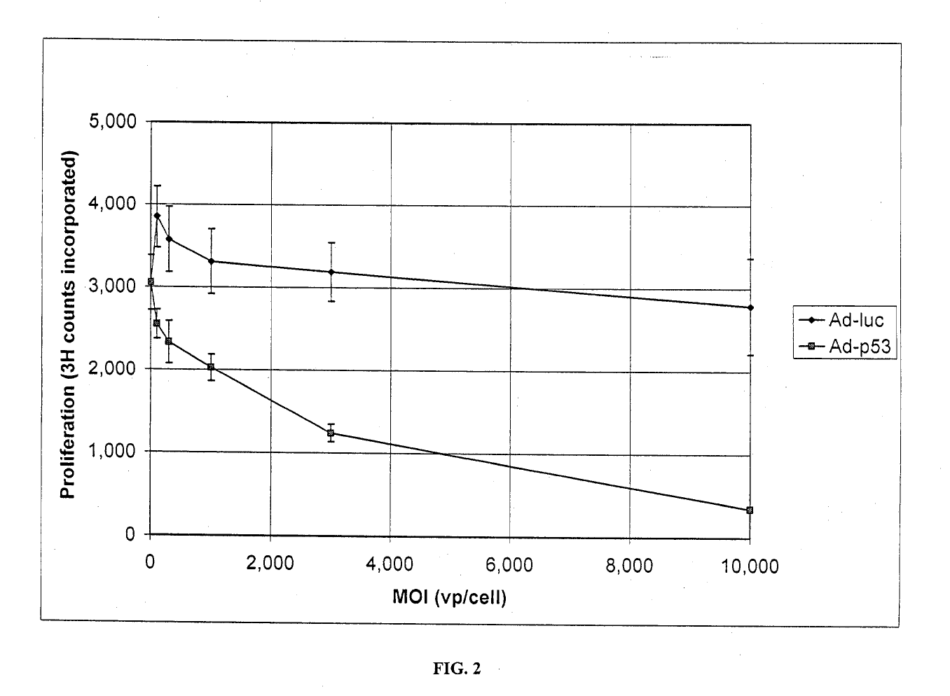

Effects on human umbilical vein endothelial cells (HUVEC). Tritiated thymidine incorporation was used to analyze the effect of INGN 201 on proliferation of HUVEC (FIG. 2). Ad5CMV-p53 infection resulted in decreased HUVEC proliferation at all MOIs tested, and in a dose dependent fashion; at 10,000 vp/cell, Ad5CMV-p53 caused an approximately 90% inhibition of proliferation, as compared to an Ad-Luc control. Ad-Luc had little or no effect.

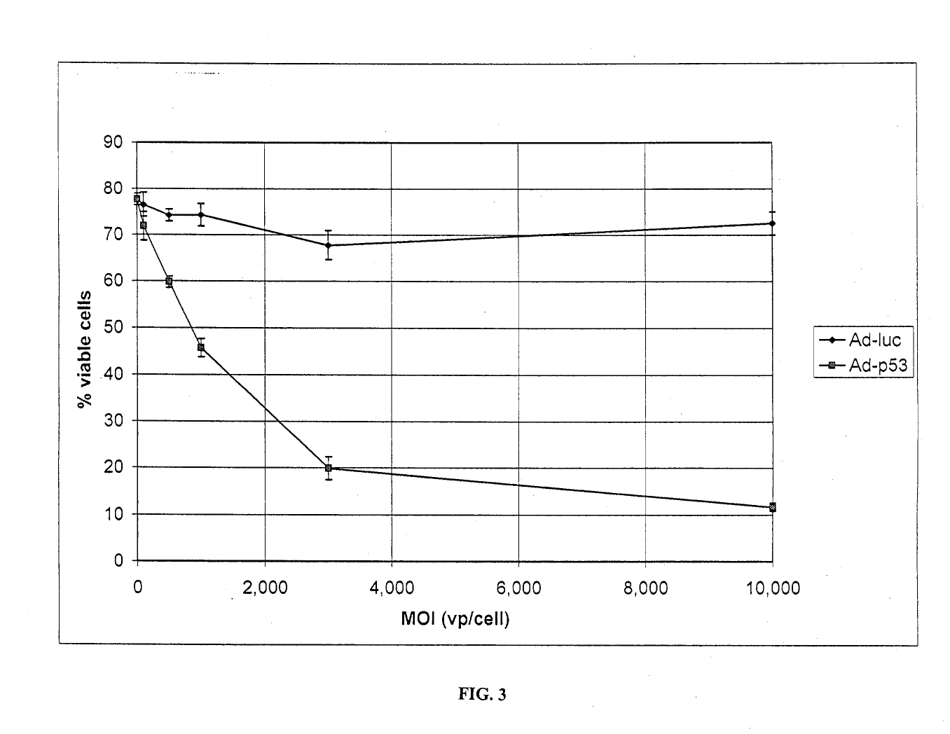

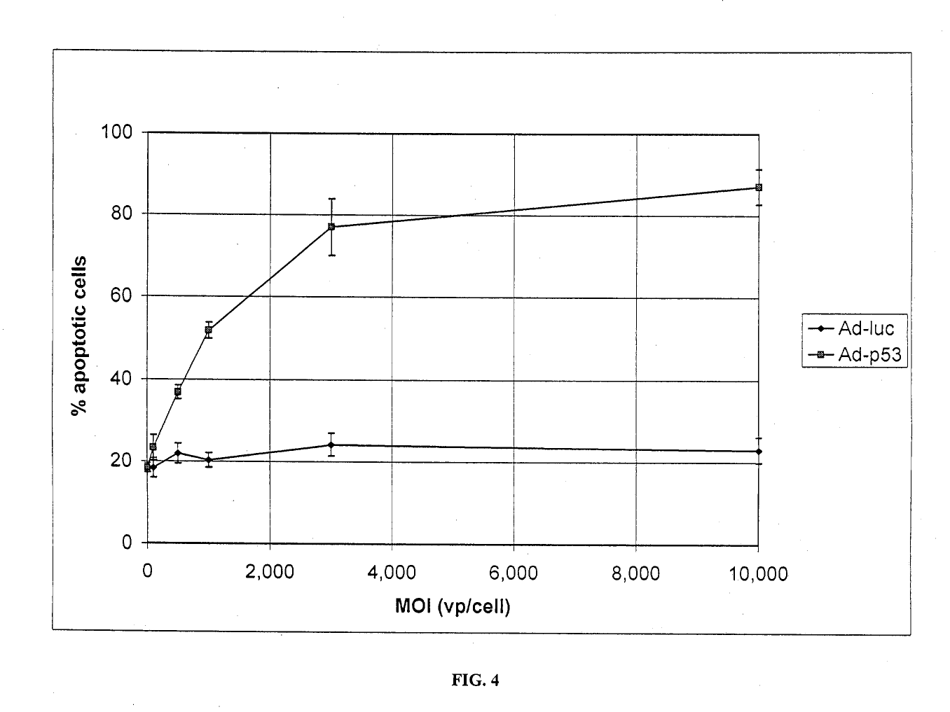

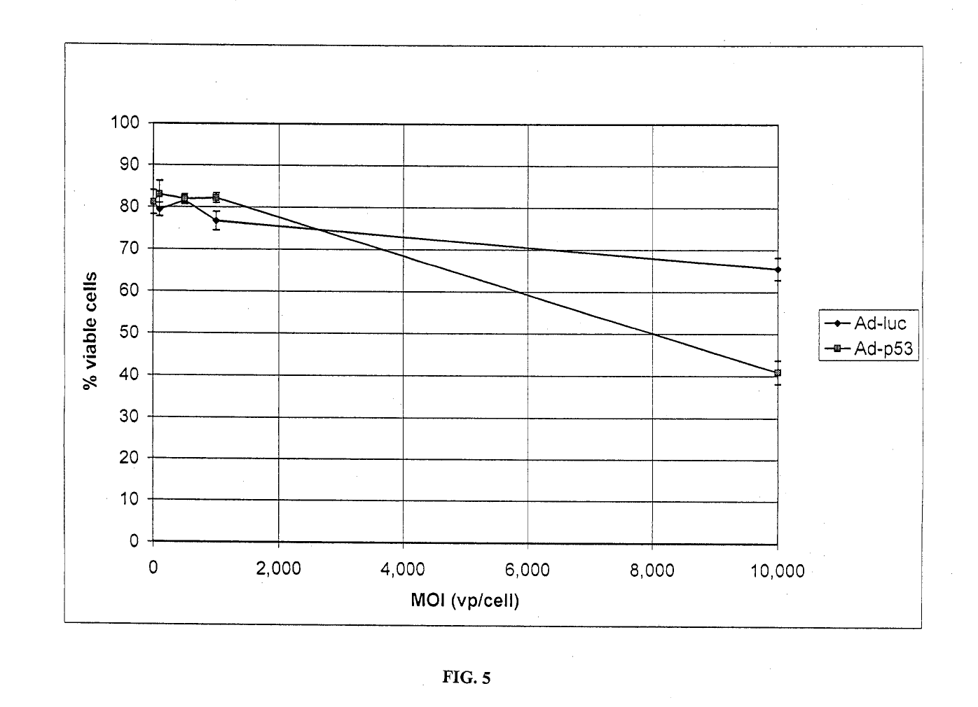

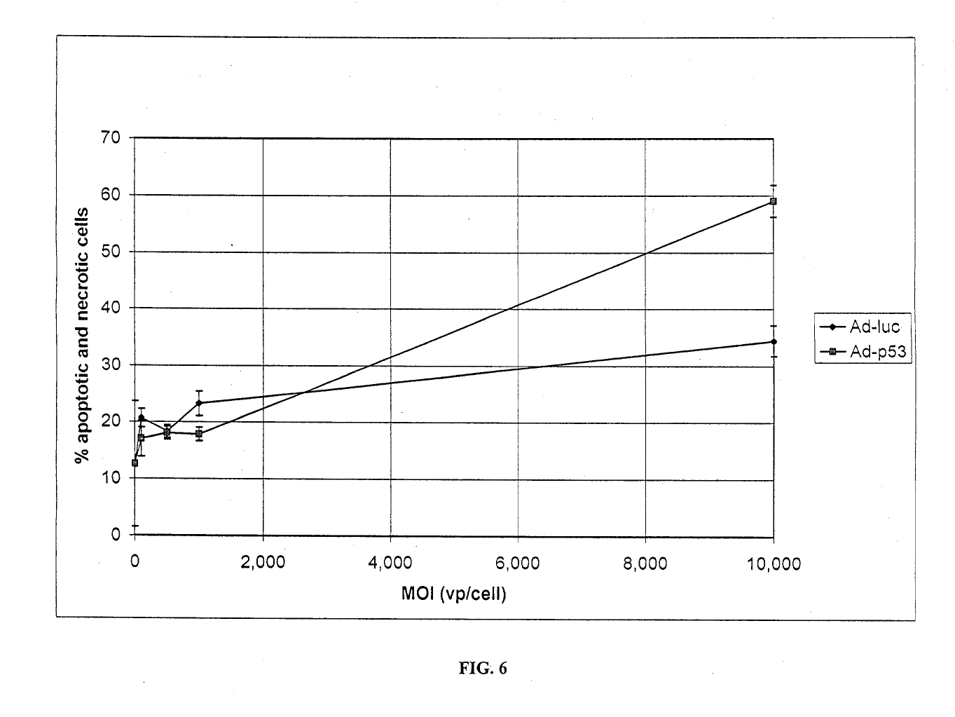

The ability of Ad5CMV-p53 to cause apoptosis and necrosis of HUVEC was assayed by flow cytometry (FACS). In one set of assays (FIG. 3 and FIG. 4), Ad5CMV-p53 caused a dose dependent decrease in the percentage of viable HUVEC ((FIG. 3) and a corresponding increase in the percentage of apoptotic cells (FIG. 4); Ad-Luc had little or no effect. However, in an independent experiment in which only attached cells were analyzed (“no floaters”), Ad5CMV-p53 caused only a small change in the percentage of viable or apoptotic cells at the highest MOI test (10,000 MOI; FIG. 5 and FIG. 6).

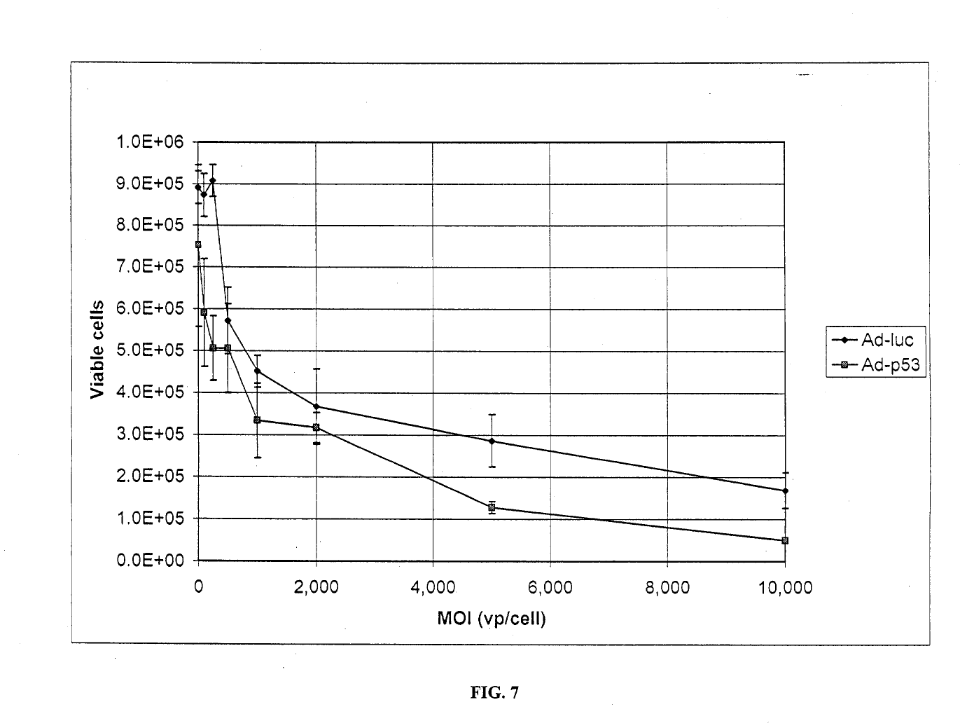

To verify the growth inhibition results were obtained from tritiated thymidine incorporation and apoptosis assays, and the viability of HUvEC was measured by their ability to exclude the dye trypan blue, after infection with Ad5CMV-p53 or Ad-Luc (FIG. 7). Both Ad-Luc and Ad5CMV-p53 caused a decrease in the number of viable cells in a dose-dependent manner. At low MOIs (<2,000 vp/cell) there was no consistent difference between Ad-Luc and Ad5CMV-p53. At the higher MOIs (>5,000 vp/cell) Ad5CMV-p53 infected HUVEC had about half the viability of Ad-Luc infected cells.

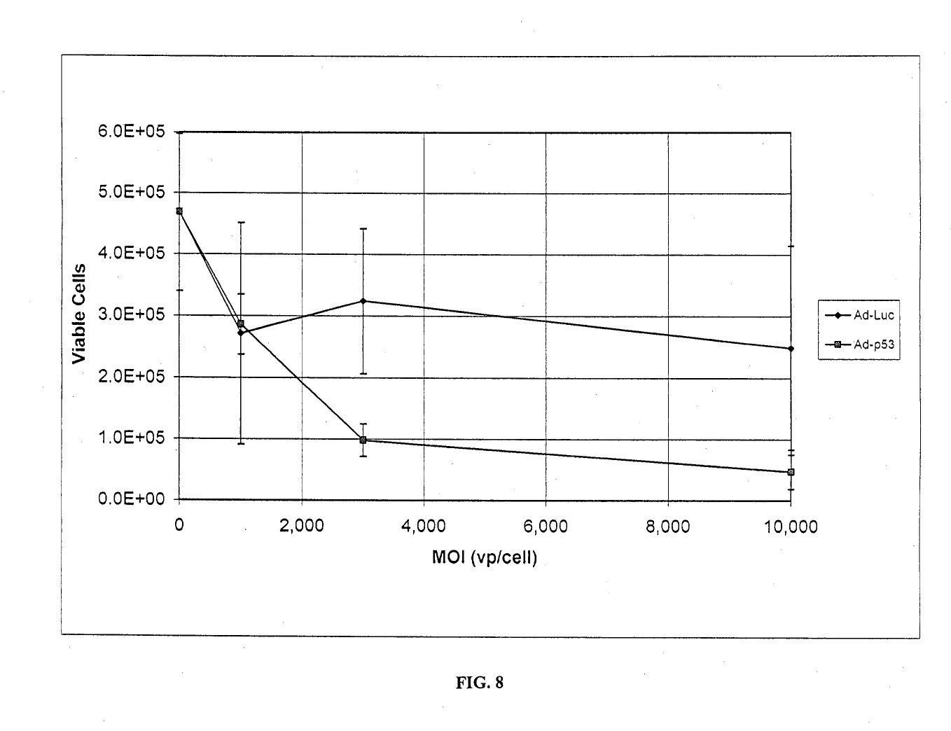

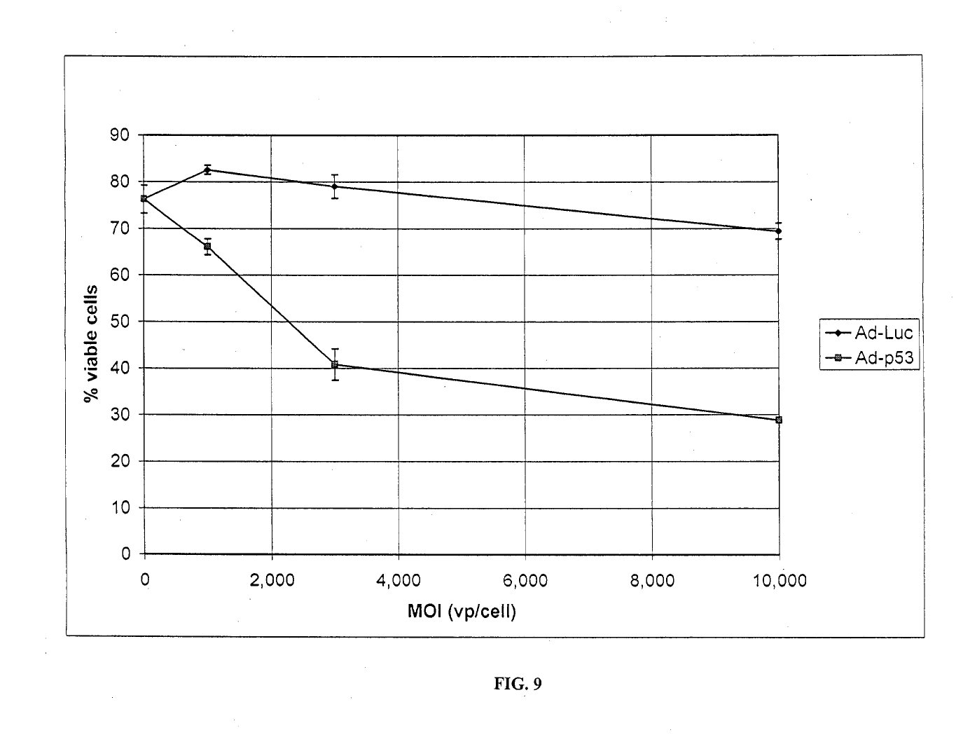

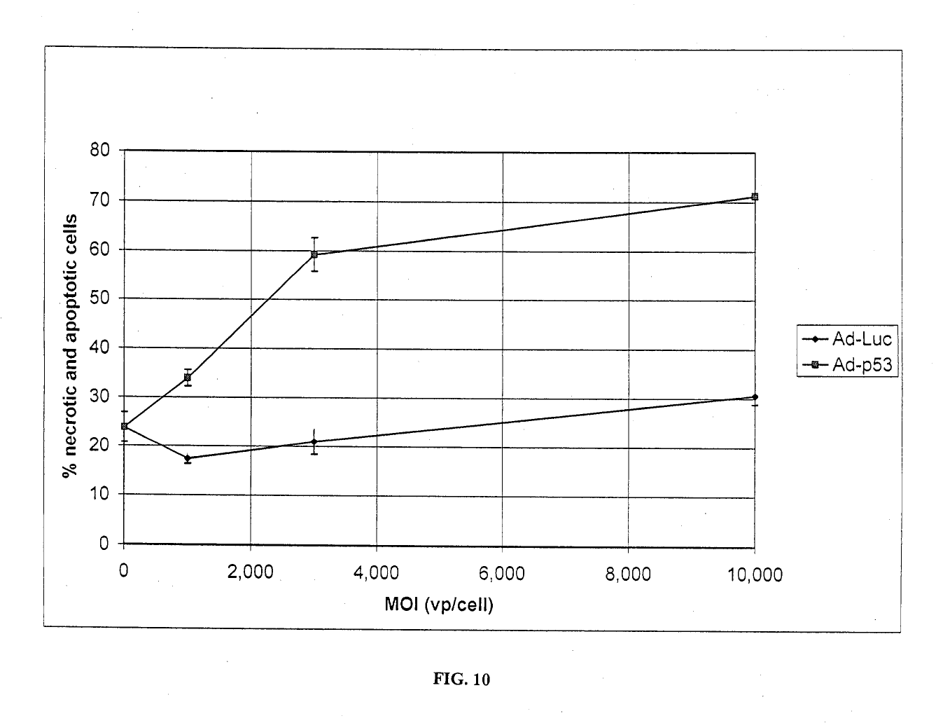

As venous or arterial endothelial cells are not usually highly proliferative in vivo, the effect of Ad5CMV-p53 on quiescent HUVEC was determined. Initial experiments showed that contact inhibition in confluent HUVEC cultures would decrease proliferation (as assayed by ³H-thymidine incorporation) yet have little effect on cell viability. After establishing quiescent HUVEC cells, they were exposed to either Ad5CMV-p53 or Ad-Luc and cell viability and apoptosis assessed. Cell viability of quiescent HUVEC (measured by trypan blue exclusion) decreased in a dose dependent fashion after Ad5CMV-p53 infection (FIG. 8), with a 90% reduction in the number of viable cells at an MOI of 10,000 vp/cell. Ad-Luc had no effect. The dose dependent decrease in viable cells as assayed by trypan blue exclusion correlated with a decrease in cell viability and an increase in apoptosis/necrosis, as assayed by FACS (FIG. 9 and FIG. 10). The decrease in cell viability caused by Ad5CMV-p53, as assayed by FACS (60%), was somewhat less than that determined by cell counting of trypan blue excluding cells (90%), but the error bars in the latter experiment were quite large.

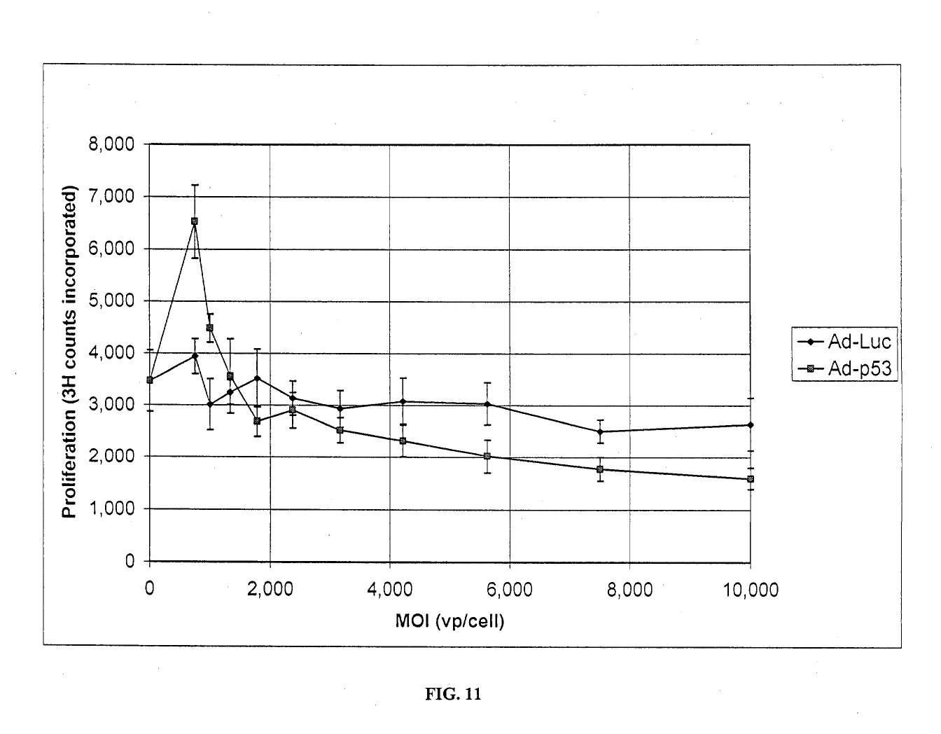

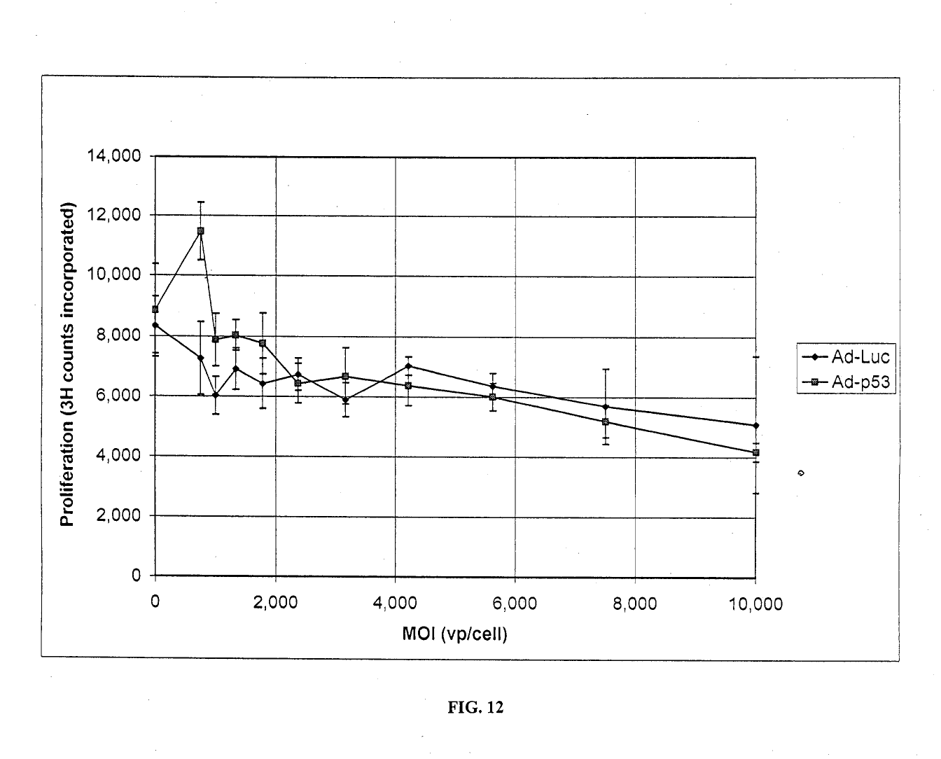

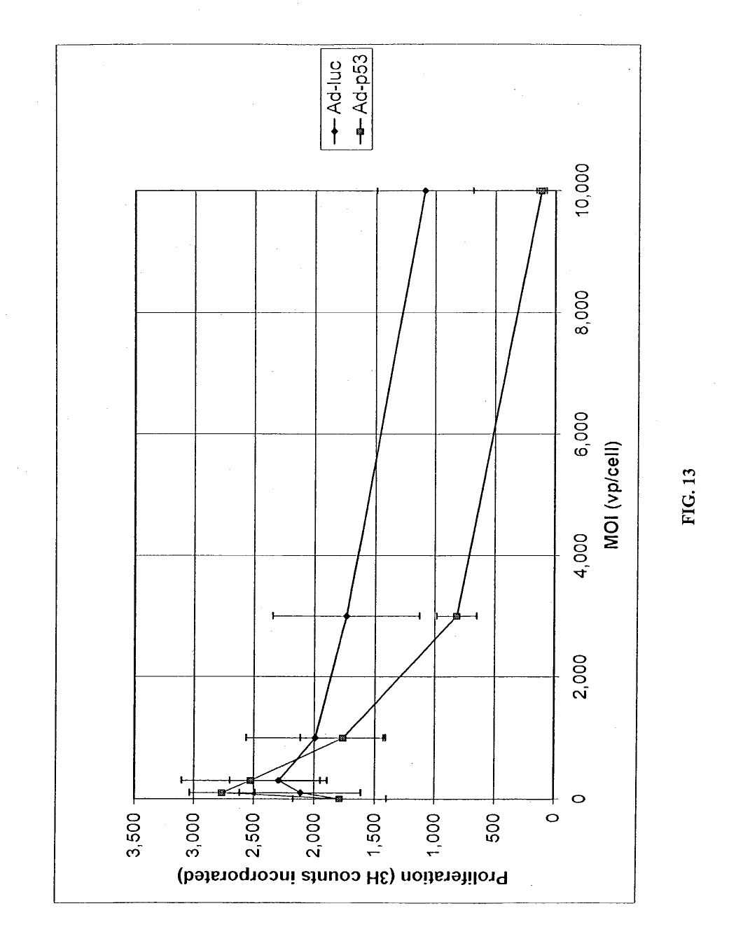

Effects on human mammary epithelial cells (HMEC). Inhibition of HMEC proliferation (³H-thymidine incorporation assay) by Ad5CMV-p53 was measured five times. The data in FIG. 11 and FIG. 12 (days 2 and 3 post-infection, respectively) is typical. There was a dose dependent decrease in proliferation caused by both Ad-Luc and Ad5CMV-p53, with little or no difference between the effects of the two vectors. The largest Ad5CMV-p53 specific inhibition of proliferation seen is shown in FIG. 13, in which the effects of Ad5CMV-p53 at MOIs of 3,000 and 10,000 vp/cell were significantly greater than those of Ad-Luc. In this experiment, a roughly 90% inhibition was seen (Ad5CMV-p53 at 10,000 vp/cell, compared to untreated); in most experiments less than 50% inhibition was seen (FIG. 11 and FIG. 12).

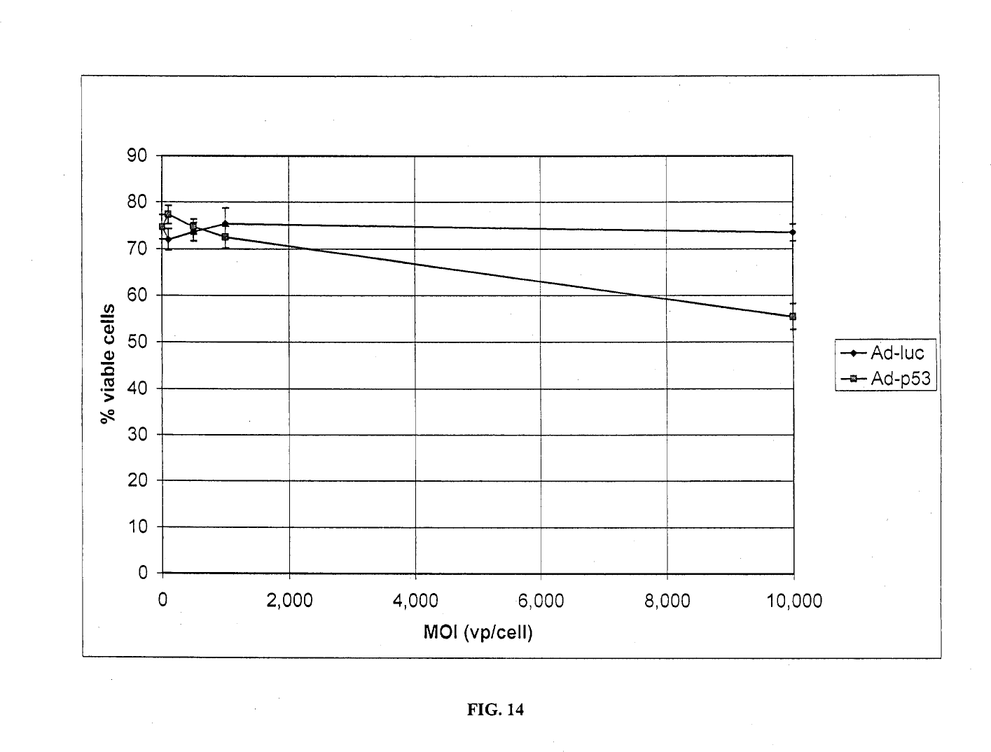

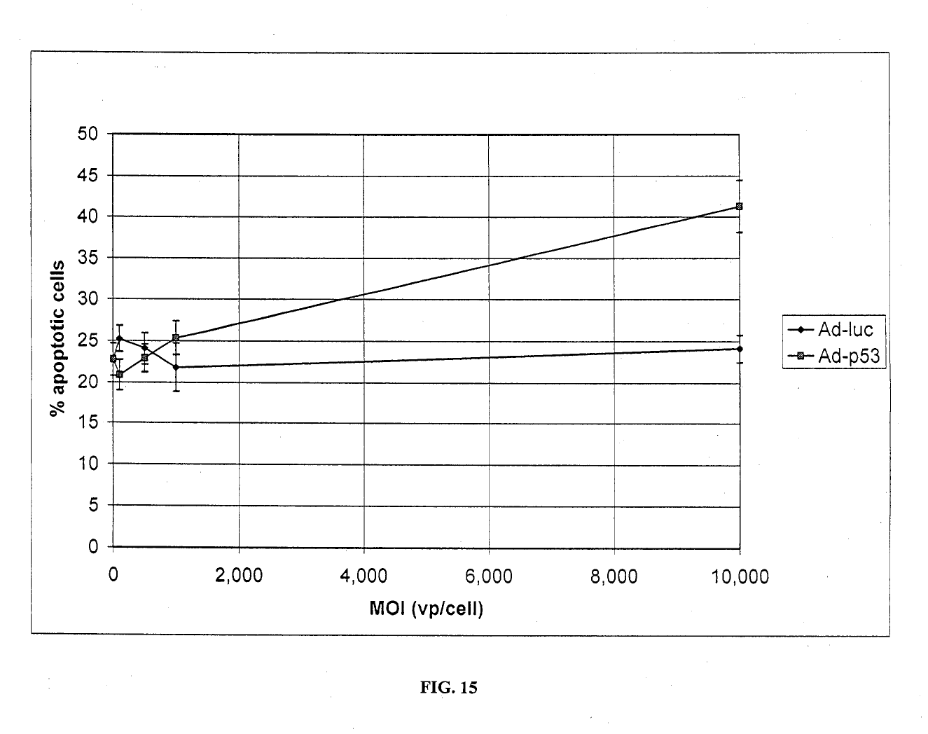

Induction of apoptosis in HMEC following Ad5CMV-p53 infection was consistent with the inhibition of proliferation (FIG. 14 and FIG. 15). No effect was seen by Ad-Luc or Ad5CMV-p53 at MOIs <1,000 vp/cell. At 10,000 vp/cell, Ad-Luc had no effect, and Ad5CMV-p53 caused a decrease in viable cells from 73% to 55%; there was a corresponding increase in the percentage of apoptotic cells.

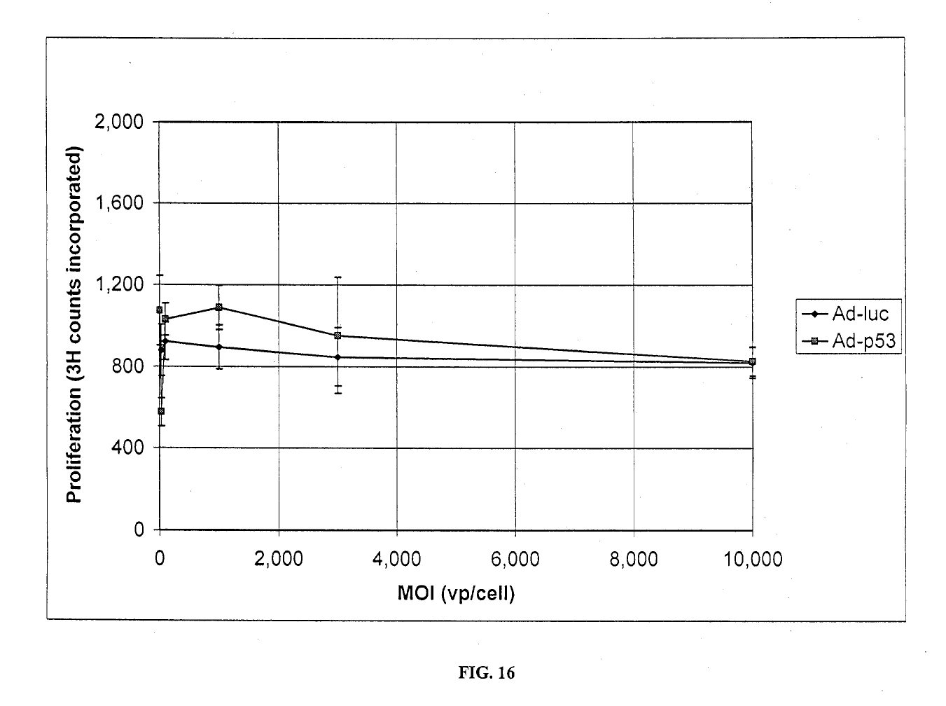

Effects on MJ90 human fibroblasts. MJ90 normal human fibroblasts were refractory to the effects of Ad5CMV-p53 or Ad-Luc (FIG. 16). A ³H-thymidine incorporation assay, at MOIs up to 10,000 vp/cell, showed no decrease in proliferation, either compared to Ad-Luc or to uninfected controls.

Effects on human coronary artery smooth muscle cells (HCASMC). The effect of Ad5CMV-p53 on two lots of HCASMC was examined; the results with the two lots were usually, but not always, consistent. These cells were grown in low serum in an attempt to induce quiescence. Preliminary experiments had shown that these cells behaved similarly when grown in high serum media.

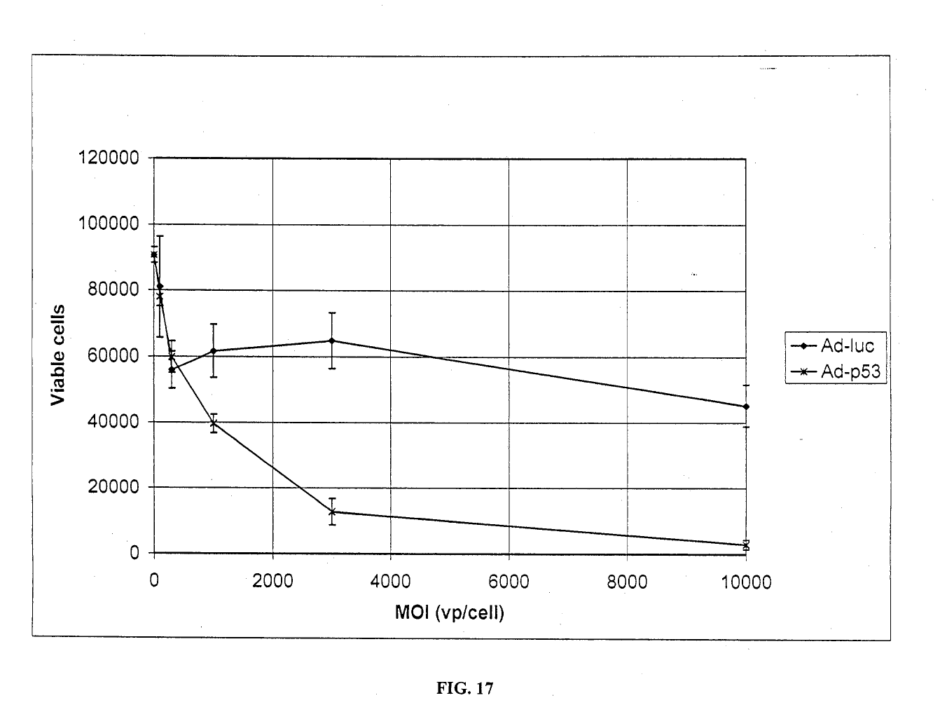

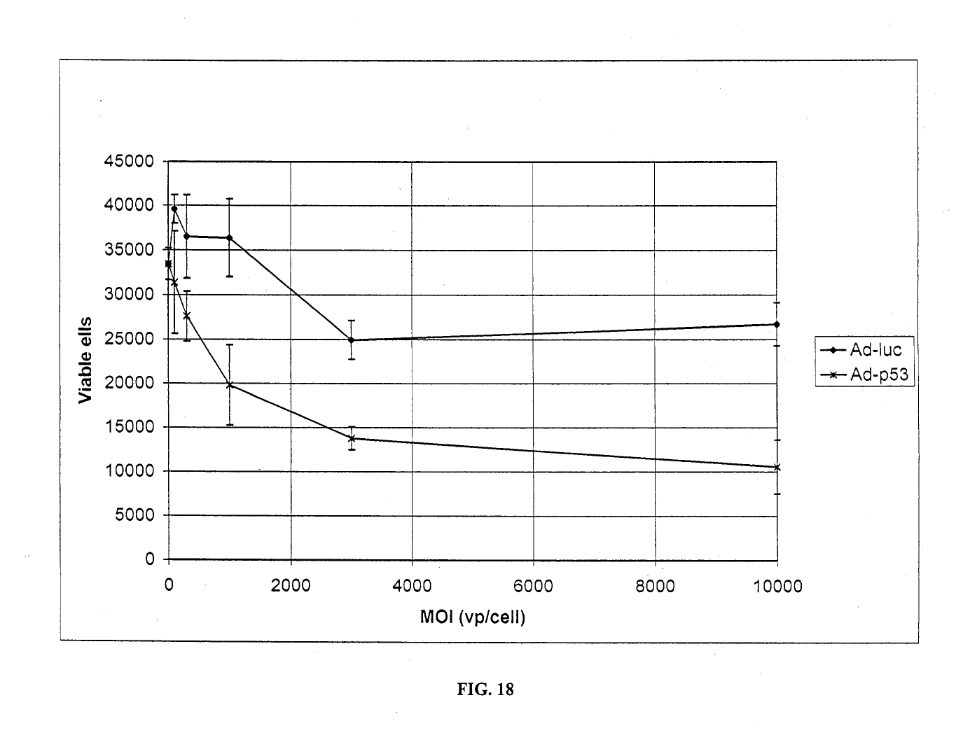

Ad5CMV-p53 caused a dose dependent decrease in the number of viable cells, while Ad-Luc had little effect (FIG. 17 and FIG. 18). While the percent decrease was different with the two lots of cells, the half maximal effect was at roughly 1,000 vp/cell. The two lots did not differ in TDE50 (FIG. 1). The data on induction of apoptosis was less clear. With lot OFI 129, a significant induction of apoptosis was found, but was not dose dependent. With lot 16445, there was a dose dependent induction of apoptosis, but the effect was slight. Another apoptosis experiment also gave equivocal results. However, it should be noted that 40% of the HCASMC were apoptotic prior to adenoviral infection.

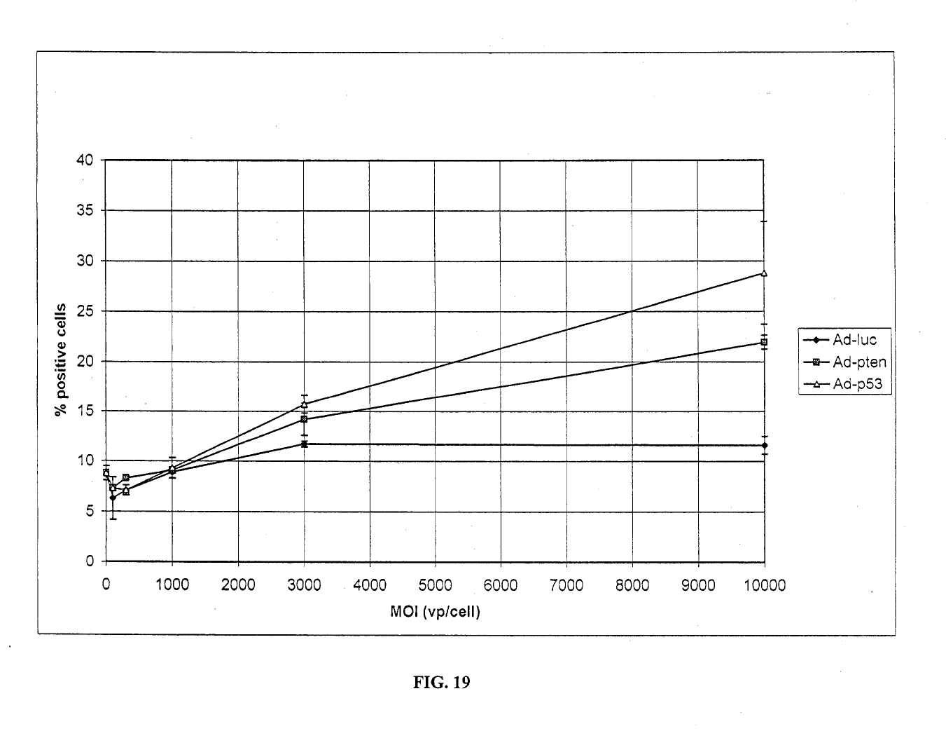

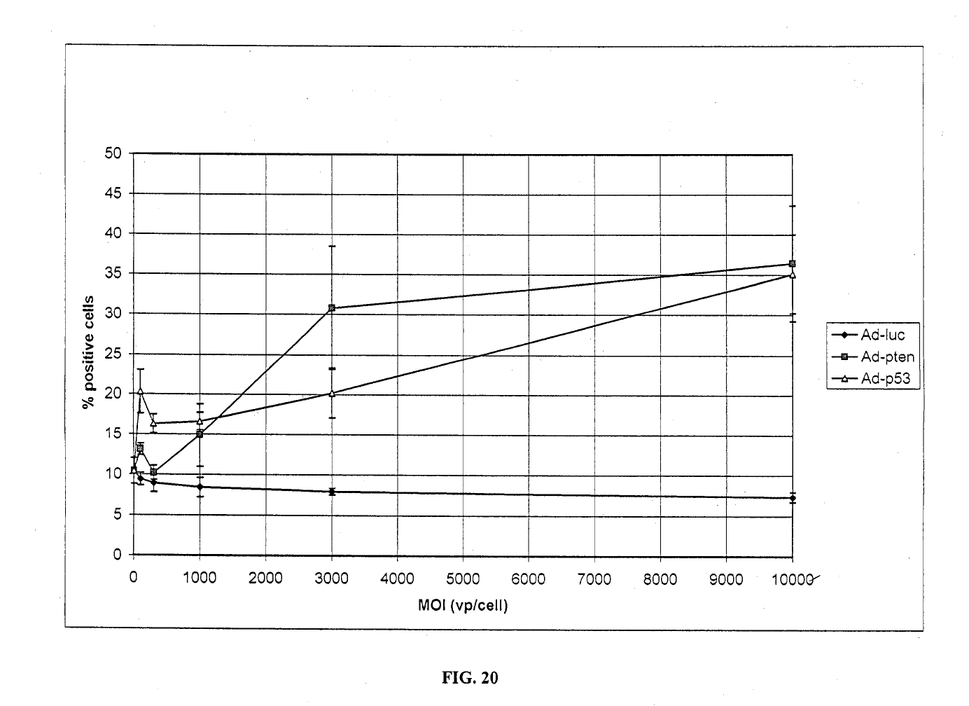

Overall, HUVEC, HMEC and HCASMC were fairly sensitive to the effects of Ad5CMV-p53. HMEC was more resistant to the effects of Ad5CMV-p53, though some non-specific adenoviral effects are seen at high infection concentrations. MJ90 fibroblasts were completely refractory to the effects of either Ad5CMV-p53 of Ad-Luc. HCASMC were highly sensitive to the apoptotic effects of Ad5CMV-p53 and in some instances were more sensitive than to Ad-PTEN (FIG. 19 and FIG. 20).

### Example 3

### Prophetic Animal Studies

The following paragraphs are a description of in vivo experiments which will demonstrate the ability of Ad5CMV-p53 or Ad-p53 to reduce neointima formation after arterial injury in atherosclerotic rabbit arteries and jugular carotid bypass vein grafts in hypercholesterolemic rabbits. These models are well established in the literature and can serve to demonstrate the efficacy of p53 in preventing narrowing of the lumen by HCASMC accumulation.

Transfermoral balloon angioplasty in Watanabe rabbits. Under approved protocols, anesthesia can be induced in 12-16-month old Watanabe rabbits of either sex with xylazine and ketamine and maintained, after endotracheal intubation, with isofluorane in oxygen. EKG and blood pressure are monitored intraoperatively. A left femoral cut-down can be performed and the femoral artery can be ligated distally. A #5F introducer sheath can be inserted into the right femoral artery through a small arteriotomy. A carotid cut-down allows for visual control of all further steps. Blood samples can be taken from the femoral artery to measure plasma TFPI concentrations and ACT, PT, aPTT. Unfractionated heparin (150 units/kg) can be given just before angioplasty. Over a 0.014 inch coronary guidewire, a 2.5 mm×20 mm balloon angioplasty catheter can be advanced under fluoroscopy to the carotid artery. The balloon can be inflated 5× to 5 atm (30 sec interval between inflations). The balloon catheter can be exchanged with a double balloon and an approximately 3 cm long carotid segment including the injured area can be isolated by inflating the 2 balloons. Side branches are tied off with suture. After flushing the isolated arterial segment, the viral vector is delivered with a 1 ml syringe, maintaining arterial pressure at about 0.5 atm for 30 min as described (Zoldhelyi et al., 2001). The injured/transduced area can be marked with non-absorbable suture. The femoral sheath is removed and the femoral artery and inguinal incision repaired. The rabbits are allowed to recover and returned to their housing after receiving buprenorphine, 0.02-0.05 mg/kg, as analgesic. Baytril, 5 mg/kg subcutaneously, can be given daily for one week as antibiotic.

The rabbits are sacrificed at various intervals as indicated below, using 2 mL of Beuthanasia-D. For histomorphometry, the animals are initially sacrificed 4 weeks after surgery and the arteries are pressure-perfused at 100 mm Hg with 10% neutral buffered formaldehyde. Samples from liver, lung, kidney, and heart can be frozen for later PCR analysis for the presence of recombinant virus using specific primers targeting the CMV promoter (forward) and foreign gene (reverse).

Dose-finding study. Evidence has accumulated that a viral titer of 4-5×10 ⁹ PFU/mL or higher induces endothelial activation, inflammation, and possibly acceleration of atherogenesis uninjured or minimally injured arteries. In contrast, the inventors and others have not seen acceleration of neointima formation or thrombosis when recombinant adenoviral vectors were applied to severely injured (and endothelium denuded) arteries.

Proof of gene transfer in injured artery can be sought after sacrifice, 4 days after surgery, by immunohistochemistry, or where this is negative, by immunoblotting, and by demonstration of p53 mRNA. The inventors will conduct studies with matched titers of Ad5CMV-p53, Ad-p53, and Ad-Luciferase (Ad-Luc). Using Ad5CMV-p53 and Ad-p53 at a dose resulting in local p53 (over)expression, 39 animals will be studied for the efficacy of Ad5CMV-p53 and Ad-p53, to prevent stenosis of the injured vessel wall.

The following parameters can be measured and calculated on each injured arterial section: outer circumference, lumen area, intima and media thickness, intima/media ratio. The neointima is distinguished by its position relative to the internal elastic lamina.

Use of p53 for the prevention of intimal thickening of other vascular conduits. Autologous venous vessels remain the main source of vascular tissue used for bypass grafting in surgery for severe coronary artery disease. Three out of four bypass conduits are constructed from saphenous or other veins. The mid- and long-term success after coronary bypass surgery, however, is often limited by the development of bypass vein graft disease. Several studies have shown that about fifty percent of vein bypass grafts are occluded within 10 years, which represents a major issue for patients who are forced to undergo repeat surgical procedures or, in case of inoperability, have only limited options for revascularization. The underlying cellular processes, which initiate bypass vain graft disease, occur in the early postoperative period. Therefore, different approaches to attenuate the chronic progress of vain graft deterioration focus on the perioperative time period.

Construction of bypass vein grafts. Thirty nine New Zealand white rabbits undergo right common carotid artery bypass grafting using the ipsilateral external jugular vein. After harvest, the jugular vein is incubated ex vivo with an adenoviral suspension of Ad5CMV-p53, Ad-p53, the control vector, Ad-Luc (or viral suspension buffer alone). A concentration of 6.5×10¹¹ plaque forming units per ml (PFU/ml) or other optimal dose is used. After a 30 minute incubation time, the vein is engrafted in reversed fashion to the right carotid artery. Restored blood flow through the graft will be confirmed in all cases by Doppler flow measurements (Transonic, Ithaca, N.Y.).

After surgery, the rabbits are placed on cholesterol-enriched chow (1%) to promote the development of hypercholesterolemia and accelerated atherosclerosis in the graft. Cholesterol blood levels and body weights are measured at the time of graft construction (baseline) and sacrifice. Four weeks after surgery, the vein grafts and the ungrafted contralateral jugular veins are perfusion-fixed with 10% formalin at 100 mm Hg. Vessels are embedded in paraffin, sectioned every 400 μm throughout the length of the graft, and stained with hematoxylin/eosin and a Verhoeft-Van Gieson elastic stain. Usually, 8-10 rings per vein graft and 2 rings of the untreated contralateral jugular vein are assessed. Magnified images are captured using an Axiophot microscope (Carl Zeiss, Inc., Thorwood, N.Y.) and digital camera (Lumina Leaf Systems, Southboro, Mass.). Images are processed with software from Optima Imaging Analysis Systems (version 6.5; Silverspring, Md.) and analyzed by a technician in blinded fashion.

The following parameters are measured and calculated on each vein graft section: outer circumference, lumen area, intima and media thickness, intima/media ratio. The media is differentiated from the adventitia by the pink staining of adventitial collagen. The neointima is distinguished by its position relative to the internal elastic lamina and by the “whirl-type” organization of cells compared to the circumferential arrangement of medial VSMC.

Assessment of cell proliferation. Balloon-Injured arteries and grafts sections are stained with the monoclonal antibody, MIB-1 (Immunotech Co., Marseille, France), which detects a nuclear antigen, Ki-67, expressed in dividing but not in growth-arrested cells. Nuclear staining is quantitatively analyzed with a Cellular Analysis System (CAS, BD Biosystems, San Diego, Calif.). Representative intima and media sections from both vein graft and contralateral vein are measured and compared. The number of immunostained nuclei versus the total nuclear count in the field of view is typically expressed as a percentage to give a proliferation index for each artery or graft and each individual tissue layer.

Statistical analysis. Data is obtained from all Ad5CMV-p53 and Ad-p53-treated animals and from animals in the 2 control groups. Results are expressed as mean values for the different animals in each group±SD. Comparisons will be made using the General Estimation Equation for repeated, correlated measurements (Zoldhelyi et al, 2001). Differences are considered statistically significant when p<0.05.

### Example 4

### Inhibition of Tube Formation

The inventors investigated the effect of adenovirus constructs on endothelial cell differentiation (tube formation). For this experiment, HUVECs were seeded on 1% gelatin coated plates and incubated at 37° C. until the cells were 70-80% confluent. After incubation, the cells were infected for 4 hours with Ad-Luc and Ad-p53 at 3 MOI's (1000, 3000, 10000 vp/cell) in basal medium. Supplemented median was added to the wells after the infection in order to dilute the virus. Twenty-four hours after infection, the media was removed and fresh supplemented media was added. After 30-35 hours post-infection, the infected cells were harvested, counted, and added to matrigel coated 48 well plates (65,000 cells/well). Test compounds and controls (media alone and Suramin 50 μM) were maintained at 37° C. for 24 hours. At the end of the incubation period, all cultures were fixed with 10% formalin. Differentiation (tube formation) was examined using an Olympus 1X-70 inverted microscope at 4× and 10× bright-field magnification. Photographs in CD format were obtained from representative wells.

Infection of endothelial cells with the adenovirus vectors resulted in some degree of cytotoxicity as evidenced by rounded, floating cells in the culture dishes. These cells were removed and only cells that remained adherent to the plate were used in the assay. The degree of cytotoxicity with Ad-luc was significantly lower than that of the Ad-p53; whereas, Ad-p53 at 10000 vp/cell killed all cells in the dish and was not able to be tested in this assay. Under the experimental conditions described, Ad-p53 at 1000 and 3000 vp/cell caused complete inhibition of tube formation. Ad-luc at 1000, 3000, and 10000 vp/cell did not cause inhibition of tube formation.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

U.S. Pat. No. RE33,925

U.S. Pat. No. 3,945,052

U.S. Pat. No. 4,047,252

U.S. Pat. No. 4,323,071

U.S. Pat. No. 4,355,426

U.S. Pat. No. 4,411,055

U.S. Pat. No. 4,439,185

U.S. Pat. No. 4,441,215

U.S. Pat. No. 4,459,252

U.S. Pat. No. 4,487,567

U.S. Pat. No. 4,550,447

U.S. Pat. No. 4,601,718

U.S. Pat. No. 4,647,416

U.S. Pat. No. 4,683,202

U.S. Pat. No. 4,695,280

U.S. Pat. No. 4,808,158

U.S. Pat. No. 4,816,028

U.S. Pat. No. 4,892,539

U.S. Pat. No. 4,909,979

U.S. Pat. No. 4,932,959

U.S. Pat. No. 4,950,238

U.S. Pat. No. 4,955,899

U.S. Pat. No. 4,969,896

U.S. Pat. No. 4,997,440

U.S. Pat. No. 5,024,671

U.S. Pat. No. 5,041,089

U.S. Pat. No. 5,059,211

U.S. Pat. No. 5,104,402

U.S. Pat. No. 5,123,917

U.S. Pat. No. 5,127,919

U.S. Pat. No. 5,156,619

U.S. Pat. No. 5,176,395

U.S. Pat. No. 5,176,661

U.S. Pat. No. 5,192,310

U.S. Pat. No. 5,197,976

U.S. Pat. No. 5,197,977

U.S. Pat. No. 5,246,452

U.S. Pat. No. 5,252,479

U.S. Pat. No. 5,256,146

U.S. Pat. No. 5,282,846

U.S. Pat. No. 5,282,848

U.S. Pat. No. 5,314,472

U.S. Pat. No. 5,358,493

U.S. Pat. No. 5,370,683

U.S. Pat. No. 5,385,580

U.S. Pat. No. 5,395,335

U.S. Pat. No. 5,405,380

U.S. Pat. No. 5,413,598

U.S. Pat. No. 5,443,498

U.S. Pat. No. 5,453,084

U.S. Pat. No. 5,464,450

U.S. Pat. No. 5,466,242

U.S. Pat. No. 5,472,418

U.S. Pat. No. 5,496,364

U.S. Pat. No. 5,500,013

U.S. Pat. No. 5,507,766

U.S. Pat. No. 5,509,931

U.S. Pat. No. 5,531,719

U.S. Pat. No. 5,551,954

U.S. Pat. No. 5,554,118

U.S. Pat. No. 5,569,200

U.S. Pat. No. 5,591,195

U.S. Pat. No. 5,607,464

U.S. Pat. No. 5,609,624

U.S. Pat. No. 5,609,626

U.S. Pat. No. 5,626,602

U.S. Pat. No. 5,628,786

U.S. Pat. No. 5,634,941

U.S. Pat. No. 5,653,745

U.S. Pat. No. 5,669,936

U.S. Pat. No. 5,672,344

U.S. Pat. No. 5,676,670

U.S. Pat. No. 5,693,745

U.S. Pat. No. 5,700,287

U.S. Pat. No. 5,713,917

U.S. Pat. No. 5,741,274

U.S. Pat. No. 5,747,128

U.S. Pat. No. 5,755,707

U.S. Pat. No. 5,769,883

U.S. Pat. No. 5,771,895

U.S. Pat. No. 5,776,096

U.S. Pat. No. 5,782,809

U.S. Pat. No. 5,800,512

U.S. Pat. No. 5,824,047

U.S. Pat. No. 5,827,327

U.S. Pat. No. 5,837,534

U.S. Pat. No. 5,840,240

U.S. Pat. No. 5,849,036

U.S. Pat. No. 5,851,230

U.S. Pat. No. 5,866,217

U.S. Pat. No. 5,871,536

U.S. Pat. No. 5,880,090

U.S. Pat. No. 5,891,195

U.S. Pat. No. 5,899,882

U.S. Pat. No. 5,910,168

U.S. Pat. No. 5,919,233

U.S. Pat. No. 5,925,061

U.S. Pat. No. 5,925,565

U.S. Pat. No. 5,928,906

U.S. Pat. No. 5,935,819

U.S. Pat. No. 5,951,539

U.S. Pat. No. 5,989,287

U.S. Pat. No. 5,994,136

U.S. Pat. No. 5,994,136

U.S. Pat. No. 5,997,573

U.S. Pat. No. 6,013,516

U.S. Pat. No. 6,015,422

U.S. Pat. No. 6,015,711

U.S. Pat. No. 6,019,788

U.S. Pat. No. 6,036,724

U.S. Pat. No. 6,053,940

U.S. Pat. No. 6,102,918

U.S. Pat. No. 6,120,532

U.S. Pat. No. 6,152,912

U.S. Pat. No. 6,162,247

U.S. Pat. No. 6,165,209

U.S. Pat. No. 6,168,620

U.S. Pat. No. 6,179,825

U.S. Pat. No. 6,187,035

U.S. Pat. No. 6,210,422

U.S. Pat. No. 6,217,566

U.S. Pat. No. 6,234,971

U.S. Pat. No. 6,241,746

U.S. Pat. No. 6,253,768

U.S. Pat. No. 6,267,777

U.S. Pat. No. 6,267,834

U.S. Pat. No. 6,290,689

U.S. Pat. No. 6,293,968

Arap et al., Cancer Res., 55(6):1351-1354, 1995.

Ausubel et al., In: Current Protocols in Molecular Biology, John, Wiley & Sons, Inc, New York, 1994.

Bacchetta et. al., In: Construction and Characterization of the Ad5CMV-p53 Vector (RPR/INGN 201), RPR Report 1999.

Baichwal and Sugden, In: Gene Transfer, Kucherlapati (ed.), New York, Plenum Press, 117-148, 1986.

Baker et al., J Clin. Invest., 101:1478-1487, 1998.

Balko, J. Surg. Res., 40:305-09, 1986.

Batterson and Roizman, J Virol., 46(2):371-377, 1983.

Benvenisty and Neshif, Proc. Natl. Acad. Sci. USA, 83(24):9551-9555, 1986.

Berns and Bohensky, Adv. Virus Res., 32:243-306, 1987.

Berns and Giraud, Curr. Top. Microbiol. Immunol., 218:1-23, 1996.

Berns, Microbiol Rev, 54(3):316-329, 1990.

Blagosklonny and El-Deiry, Int. J Cancer, 75(6):933-940, 1998.

Blomer et al., J. Virol., 71(9):6641-6649, 1997.

Burbage et al., Leuk Res., 21(7):681-690, 1997.

Bussemakers et al., Biochem. Biophys. Res. Commun., 182(1):318-324, 1992.

Caldas et al., Nat. Genet., 8(1):27-32, 1994.

Cardone et al., Science, 282:1318-1321, 1998.

Casey et al., Oncogene, 6(10):1791-1797, 1991.

Chandler et al., Proc. Natl. Acad. Sci. USA, 94(8):3596-601, 1997.

Channon and Annex, Curr. Cardiol. Rep., 2:34-38, 2000.

Cheng et al., Cancer Res., 54(21):5547-5551, 1994.

Cheung et al., J Biol. Chem., 268(32):24303-24310, 1993.

Clayman et al., J Clin. Oncol., 16(6): 2221-32, 1998.

Coffin, In: Virology, Fields et al. (Eds.), Raven Press, NY, 1437-1500, 1990.

DeLuca et al., J Virol., 56(2):558-570, 1985.

D'Orazi et al., J Gene Med., 2(1):11-21, 2002.

Dubensky et al., Proc. Natl. Acad. Sci. USA, 81:7529-7533, 1984.

Edelman and Crossin, Annu. Rev. Biochem., 60:155-90, 1991.

Ehsan and Mann, Vasc. Med., 5:103-114,2000.

Elroy-Stein et al., Proc. Natl. Acad. Sci. USA, 86(16):6126-6130, 1989.

Frixen et al., J Cell Biol., 113(1):173-185, 1991.

Fumari et al., Cancer Res., 58:5002-5008, 1998.

George et al., Human Gene Therapy 9:867-877, 1998a.

George et al., Gene Therapy, 5:1552-1560, 1998b.

George et al., Circulation, 101:296-304, 2000.

George et al., European Heart J, 17:P1062 (abstract), 1996.

George et al., Cardiovasc. Res., 33:447-459, 1997.

Ghosh and Bachhawat, In: Liver Diseases, Targeted Diagnosis and Therapy Using Specific Receptors and Ligands. Wu et al. (Eds.), Marcel Dekker, NY, 87-104, 1991.

Giancotti and Ruoslahti, Cell, 60(5):849-859, 1990.

Glorioso et al., Mol. Biotechnol., 4(1):87-99, 1995.

Gomez-Foix et al., J Biol. Chem., 267:25129-25134, 1992.

Gonzalez-Zulueta et al., Cancer Res., 55(20):4531-4535, 1995.

Graham and Prevec, Biotechnology, 20:363-390, 1992.

Graham et al., J Gen. Virl., 36(1):59-74,1977.

Granhaus et al., Seminar in Virology, 200(2):535-546, 1992.

Harui et al., J Virol., 73(7):6141-6146, 1999.

Herman et al., Cancer Res., 55(20):4525-4530, 1995.

Herz and Gerard, Proc. Natl. Acad. Sci. USA, 90:2812-2816, 1993.

Hillgenberg et al., J Virol., 75(20):9896-908, 2001.

Holland and Holland, J Biol. Chem., 255(6):2596-2605, 1980.

Hollstein et al., Science, 253(5015):49-53, 1991.

Hones and Roizman, J Virol., 16(5):1308-1326, 1975.

Hones and Roizman, J Virol., 14(1):8-19, 1974.

Hussussian et al., Nat. Genet., 8(1):15-21, 1994.

Inouye and Inouye, Nucleic Acids Res., 13:3101-3109, 1985.

Jung et al., Cardiovasc. Res., 48:148-157, 2000.

Kamb et al., Nat. Genet., 8(1):23, 1994.

Kaneda et al., Science, 243:375-378, 1989.

Kato et al., J Biol. Chem., 266:3361-3364, 1991.

Kawabe et al., Int. J Radiat. Biol., 77(2):185-194, 2001.

Kearns et al., Gene Ther., 3(9):748-755, 1996.

Kim et al., Int. J Oncol., 18(2):241-247, 2001.

Kim et al., J Virol., 76(4)1892-1903, 2002.

Kotin and Bems, Virology, 170(2):460-467, 1989.

Kotin et al., Genomics, 10(3):831-834, 1991.

Kotin et al., Proc. Natl. Acad. Sci. USA, 87(6):2211-2215, 1990.

Le Gal La Salle et al., Science, 259:988-990, 1993.

Levrero et al., Gene, 101: 195-202, 1991.

Li and Sun, Proc. Natl. Acad. Sci. USA, 95:15406-15411, 1988.

Li and Sun, Cancer Res., 57(11):2124-2129, 1997.

Li et al., Science, 275(5308):1876-1878, 1997.

Lidor et al., Am. J Obstet. Gynecol., 177(3):579-585, 1997.

Lin and Guidotti, J Biol. Chem., 264:14408-14414, 1989.

Liu and Gazitt, Exp. Hematol., 28(12):1354-1362, 2000.

Macejak and Sarnow, Nature, 353:90-94, 1991.

Maehama and Dixon, J. Biol. Chem., 273:13375-13378, 1998.

Maizel et al., Virology, 36:115-125, 1968.

Maniatis, et al., Molecular Cloning, A Laboratory Manual, Cold Spring Harbor Press, Cold Spring Harbor, N.Y., 1989.

Mann, Curr. Cardiol. Rep., 2:29-33, 2000.

Mann et al., Cell, 33:153-159, 1983.

Massuda et al., Proc. Natl. Acad. Sci. USA, 94(26):14701-14706, 1997.

Matsura Biochim. Biophys. Acta, 1123(3):309-315, 1992.

Merlo et al., Nat. Med., 1(7):686-692, 1995.

Mizukami et al., Virology, 217(1):124-130, 1996.

Myers et al., Proc. Natl. Acad. Sci. USA, 95:13513-13518, 1998.

Myers et al., Proc. Natl. Acad. Sci. USA, 94:9052-9057, 1997.

Naldini et al., Science, 272(5259):263-267, 1996.

Nicolas and Rubinstein, In: Vectors: A survey of molecular cloning vectors and their uses, Rodriguez and Denhardt (Eds.), Stoneham: Butterworth, 494-513, 1988.

Nicolau et al., Methods Enzymol., 149:157-176, 1987.

Nobori et al., Nature, 368(6473):753-756, 1994.

Obrink, Bioessays, 13(5):227-234, 1991.

Odin and Obrink, Exp. Cell Res., 171(1):1-15, 1987.

Okamoto et al., Proc. Natl. Acad. Sci. USA, 1(23):11045-11049, 1994.

Oren, J Biol. Chem., 274:51:36031-36034, 1999.

Orlow et al., Cancer Res., 54(11):2848-2851, 1994.

Ostrove et al., Virology, 113(2):521-33, 1981.

Paskind et al., Virology, 67:242-248, 1975.

PCT/GB96/02588

PCT/US00/14350

PCT/US99/05781

Pelletier and Sonenberg, Nature, 334(6180):320-325, 1988.

Ponnazhagan et al., J Gen. Virol., 77(6):1111-1122, 1996.

Ponnazhagan et al., J Virol., 71(4):3098-3104, 1997.

Post et al., Cell, 24(2):555-565, 1981.

Ragot et al., Nature, 361:647-650, 1993.

Ramaswamy et al, Proc. Natl. Acad. Sci. USA, 96:2110-2115, 1999.

Remington's Pharmaceutical Sciences, 18th Ed. Mack Printing Company, 1289-1329, 1990.

Rich et al., Hum. Gene Ther., 4:461-476, 1993.

Ridgeway, In: Vectors: A survey of molecular cloning vectors and their uses. Rodriguez and Denhardt (Eds.), Stoneham: Butterworth, 467-492, 1988.

Rosenfeld et al., Science, 252:431-434, 1991.

Rosenfeld et al., Cell, 68:143-155,1992.

Roux et al., Proc. Natl. Acad. Sci. USA, 86:9079-9083, 1989.

Sambrook et. al., In: Molecular Cloning: A Laboratory Manual, 2d Ed., Cold Spring Harbor Laboratory Press, Cold Spring Harbor, N.Y., 2001.

Samulski et al., EMBO J, 10:3941-3950, 1991.

Serrano et al., Science, 267(5195):249-252, 1995.

Serrano et al., Nature, 366:704-707, 1993.

Smith and Moss, Gene, 25(1):21-28, 1983.

Smith and Walsh, Curr. Cardiol. Rep., 2:13-23, 2000.

Southgate et al., Arterioscler. Thromb. Vasc. Biol., 19(7):1640-1649, 1999.

Steck et al., Nat. Genet., 15:356-62, 1997.

Stratford-Perricaudet and Perricaudet, In: Human Gene Transfer, Cohen-Haguenauer and M. Boiron (Eds.,), John Libbey Eurotext, France, 51-61, 1991.

Stratford-Perricaudet et al., Hum. Gene. Ther., 1:241-256, 1990.

Takahasi and Sawasaki, In Vitro Cell Dev. Biol., 28A(6):380-382, 1992.

Tamura et al., Science 280:1614-1617, 1998.

Temin, In: Gene Transfer, Kucherlapati (ed.), NY, Plenum Press, 149-188, 1986.

Tian et al., Neuropathol. Exp. Neurol., 58:472-479, 1999.

Tonks and Myers, Science, 286:2096-2097, 1999.

Umbas et al., Cancer Res., 52(18):5104-5109, 1992.

Van Doren et al., J Virol., 50(2):606-614, 1984.

Wattanapitayakul and Bauer, Biomed. Pharmacother., 54:487-504, 2000.

Weinberg, Science, 254(5035): 1138-1146, 1991.

Wong et al., Gene, 10:87-94, 1980.

Wood et al., Cancer Gene Ther., 6(4):367-372, 1999.

Wu and Wu, Adv. Drug Delivery Rev., 12:159-167, 1993.

Yla-Herttuala and Martin, Lancet., 15(355):213-222, 2000.

Zhang et al., Biotechniques, 15(5):868-872, 1993.

Zhang et al., Hum. Gene Ther., 6(2): 155-164, 1995.

Zoldhelyi et al., Proc. Natl. Acad. Sci. USA, 98:4078-4083, 2001.

Zufferey et al., Nat. Biotechnol., 15(9):871-875, 1997.

What is claimed:

1. A method for reducing vascular smooth muscle cell (VSMC) proliferation comprising introducing into said cell a first expression cassette comprising a promoter active in said cell and a first nucleic acid segment encoding p53, wherein said nucleic acid segment is under the transcriptional control of said promoter.

2. The method of claim 1, wherein said expression cassette is comprised within a viral vector.

3. The method of claim 2, wherein said viral vector is selected from the group consisting of an adenovirus, a retrovirus, an adeno-associated virus, a vaccinia virus, a herpesvirus or a polyoma virus.

4. The method of claim 3, wherein said viral vector is an adenoviral vector.

5. The method of claim 4, wherein said adenoviral vector is a replication defective adenoviral vector.

6. The method of claim 5, wherein said replication defective adenoviral vector lacks at least a portion of the E1 region.

7. The method of claim 6, wherein said replication defective adenoviral vector does not encode functional E1A or E1B proteins.

8. The method of claim 7, wherein said replication defective adenoviral vector lacks the entire E1region.

9. The method of claim. 1, wherein said expression vector is a non-viral expression vector.

10. The method of claim 9, wherein said non-viral expression vector is encapsulated in a lipid delivery complex.

11. The method of claim 1, wherein said promoter is a constitutive promoter.

12. The method of claim 1, wherein said promoter is preferentially active in VSMC.

13. The method of claim 1, wherein said expression construct further comprises a polyadenylation signal.

14. The method of claim 1, wherein said expression construct further comprises a second nucleic acid segment encoding an anti-proliferative protein other than p53.

15. The method of claim 14, wherein said anti-proliferative protein comprises a tumor suppressor, an inducer of apoptosis, a cell cycle regulator, a tissue factor inhibitor, an inhibitor of a platelet-borne group of proliferative or pro-migratory molecules, or a toxin.

16. The method of claim 14, wherein said first and second nucleic acid segments are under the control of the same promoter.

17. The method of claim 16, wherein said first and second nucleic acid segments are separated by an internal ribosome entry site.

18. The method of claim 14, wherein said first and second nucleic acid segments are under the control of different promoters.

19. The method of claim 15, wherein said anti-proliferative protein is a tumor suppressor.

20. The method of claim 19, wherein said tumor suppressor is PTEN or Rb.

21. The method of claim 15, wherein said anti-proliferative protein is an inducer of apoptosis.

22. The method of claim 21, wherein said inducer of apoptosis is Bcl-2, Bax, Bad, or Bid.

23. The method of claim 15, wherein said anti-proliferative protein is a cell cycle regulator.

24. The method of claim 23, wherein said cell cycle regulator is E2F-1.

25. The method of claim 15, wherein said anti-proliferative protein is an enzyme.

26. The method of claim 25, wherein said enzyme is cyclooxygenase-1 (COX-1) or tissue factor pathway inhibitor (TFP-I), endothelial nitric oxide synthase (eNOS), inducible nitric oxide (iNOS), or inhibitors of matix metalloprotein (TIMP-1 or TIMP-2).

27. The method of claim 1, wherein said expression construct further comprises an origin of replication.

28. The method of claim 1, wherein said VSMC is located in a subject.

29. The method of claim 28, wherein said subject is a human.

30. The method of claim 28, wherein said VSMC are located in a vein or artery that has been or will be subject to trauma.

31. The method of claim 30, wherein said trauma comprises angioplasty.

32. The method of claim 30, wherein said trauma comprises insertion of a stent, a graft or a conduit.

33. The method of claim 28, wherein said VSMC are located in an uninjured early atherosclerotic lesion.

34. The method of claim 28, wherein said VSMC are located in the vasculature of a transplanted organ.

35. The method of claim 28, further comprising administering to said subject an anti-inflammatory compound.

36. The method of claim 28, further comprising administering to said subject an inhibitor of thrombosis.

37. The method of claim 28, further comprising administering to said subject a fibrolytic agent.

38. The method of claim 28, further comprising administering to said subject endoluminal radiation.

39. The method of claim 1, further comprising introducing into said cell a second expression cassette comprising a promoter active in said cell and a second nucleic acid segment encoding an anti-proliferative protein other than p53, wherein said nucleic acid segment is under the transcriptional control of said promoter.

40. The method of claim 39, wherein said anti-proliferative protein comprises a tumor suppressor, an inducer of apoptosis, a cell cycle regulator, a tissue factor inhibitor, an inhibitor of a platelet-borne group of proliferative or pro-migratory molecules, or a toxin.

41. The method of claim 40, wherein said anti-proliferative protein is a tumor suppressor.

42. The method of claim 41, wherein said tumor suppressor is PTEN or Rb.

43. The method of claim 40, wherein said anti-proliferative protein is an inducer of apoptosis.

44. The method of claim 43, wherein said inducer of apoptosis is Bcl-2, Bax, Bad, or Bid.

45. The method of claim 40, wherein said anti-proliferative protein is a cell cycle regulator.

46. The method of claim 45, wherein said cell cycle regulator is E2F-1.

47. The method of claim 40, wherein said anti-proliferative protein is an enzyme.

48. The method of claim 47, wherein said enzyme is cyclooxygenase-1 (COX-1) or tissue factor pathway inhibitor (TFP-I), endothelial nitric oxide synthase(eNOS), inducible nitric oxide (iNOS), or inhibitors of matix metalloprotein (TIMP-1 or TIMP-2).

49. The method of claim 1, wherein introducing comprises intravenous or intraarterial delivery of said expression cassette.

50. The method of claim 49, further comprising restricting blood flow away from the region of the delivery.

51. The method of claim 50, wherein restricting comprises applying a tourniquet or cuff to said human subject.

52. The method of claim 49, wherein said expression cassette is coated or impregnated on the surface of an intravascular device prior to insertion into said human subject.

53. A method for reducing vascular smooth muscle cell (VSMC) migration comprising introducing into said cell an expression cassette comprising promoter active in said cell and a first nucleic acid segment encoding p53, said nucleic acid segment under the transcriptional control of said promoter.

54. A method for reducing an atherosclerotic lesion in a subject comprising introducing into cells of said lesion an expression cassette comprising a promoter active in said cell and a first nucleic acid segment encoding p53, said nucleic acid segment is under the transcriptional control of said promoter.

55. A method for preventing development of an atherosclerotic lesion in a subject comprising introducing into vascular smooth muscle cells of said subject an expression cassette comprising a promoter active in said cell and a first nucleic acid segment encoding p53, said nucleic acid segment is under the transcriptional control of said promoter.

56. A method for of inhibiting intimal hyperplasia in a subject comprising introducing into a vascular smooth muscle cells of said subject an expression cassette comprising a promoter active in said cell and a first nucleic acid segment encoding p53, said nucleic acid segment is under the transcriptional control of said promoter.
